(19) United States
(12) Patent Application Publication
(10) Pub. No.: US 2025/0339139 A1
(43) Pub. Date: Nov. 6, 2025

(54) IMPLANT DELIVERY SYSTEMS

(71) Applicant: Teleflex Life Sciences LLC, Wilmington, DE (US)

(72) Inventors: Curtis Yarra, Oakland, CA (US); Alexander Gordon, San Carlos, CA (US); Ernest Cabreza, Dublin, CA (US); Erik Noel, San Ramon, CA (US); Robert Elliott DeCou, Pleasanton, CA (US); Javier Fajardo, Antioch, CA (US)

(73) Assignee: Teleflex Life Sciences LLC, Wilmington, DE (US)

(21) Appl. No.: 19/197,504

(22) Filed: May 2, 2025

Related U.S. Application Data
(60) Provisional application No. 63/641,778, filed on May 2, 2024.
(60) Provisional application No. 63/641,745, filed on May 2, 2024.
(60) Provisional application No. 63/641,550, filed on May 2, 2024.
(60) Provisional application No. 63/641,720, filed on May 2, 2024.
(60) Provisional application No. 63/641,570, filed on May 2, 2024.
(60) Provisional application No. 63/641,534, filed on May 2, 2024.
(60) Provisional application No. 63/794,675, filed on Apr. 25, 2025.

(51) Int. Cl. A61B 17/04; A61B 1/015; A61B 1/307; A61B 17/00

(52) U.S. Cl. CPC A61B 17/0401; A61B 1/015; A61B 1/307; A61B 2017/00274; A61B 2017/0409; A61B 2017/0419; A61B 2017/0464; A61B 2217/007

(57) ABSTRACT

Implant delivery systems are configured to deploy implants into targeted tissue within a patient. The targeted tissue includes at least a portion of a prostate gland, and the implants are configured to compress or retract the tissue. Implant delivery systems include handheld components. Irrigation systems control fluid inflow and outflow at the treatment site. Sheath devices deliver irrigant to a treatment site within a patient, and elongate shaft devices remove contaminated irrigant, blood, and debris. Visualization systems provide an unobstructed view of the treatment site during implant deployment.

### CROSS REFERENCE TO RELATED APPLICATIONS

This application claims priority to U.S. Provisional Patent Application No. 63/641,534, filed on May 2, 2024, titled “Implant Delivery System” and U.S. Provisional Patent Application No. 63/641,778, filed on May 2, 2024, titled “Implant Delivery System” and U.S. Provisional Patent Application No. 63/641,570, filed on May 2, 2024, titled “Implant Delivery System” and U.S. Provisional Patent Applicant No. 63/641,720, filed on May 2, 2024, titled “Implant Delivery System” and U.S. Provisional Patent Application No. 63/641,745, filed on May 2, 2024, titled “Implant Delivery System” and U.S. Provisional Patent Application No. 63/641,550, filed on May 2, 2024, titled “Implant Delivery System” and U.S. Provisional Application No. 63/794,675, filed Apr. 25, 2025, titled “Implant Delivery Systems.” The contents of these applications are hereby incorporated by reference in their entireties herein.

### TECHNICAL FIELD

The subject matter of this patent document relates to the field of medical devices. More particularly, but not by way of limitation, the subject matter relates to medical devices, systems, and methods for relieving pressure on a prostatic urethra by compressing at least a portion of a prostate gland.

### BACKGROUND

Benign Prostatic Hyperplasia (“BPH”) is one of the most common medical conditions that affect men, especially elderly men. It has been reported that, in the United States, more than half of all men have histopathologic evidence of BPH by age 60 and, by age 85, approximately 9 out of 10 men suffer from the condition. Moreover, the incidence and prevalence of BPH are expected to increase as the average age of the population in developed countries increases.

The prostate gland enlarges throughout a man's life. In some men, the prostatic capsule around the prostate gland may prevent the prostate gland from enlarging further. This causes the inner end of the prostate gland to squeeze the urethra. This pressure on the urethra increases resistance to urine flow through the end of the urethra enclosed by the prostate. Thus, the urinary bladder has to exert more pressure to force urine through the increased resistance of the urethra. Chronic over-exertion causes the muscular walls of the urinary bladder to remodel and become stiffer. This combination of increased urethral resistance to urine flow and stiffness and hypertrophy of urinary bladder walls leads to a variety of lower urinary tract symptoms (LUTS) that may severely reduce the patient's quality of life. These symptoms include weak or intermittent urine flow while urinating, straining when urinating, hesitation before urine flow starts, feeling that the bladder has not emptied completely even after urination, dribbling at the end of urination or leakage afterward, increased frequency of urination particularly at night, urgent need to urinate etc.

In addition to patients with BPH, LUTS may also be present in patients with prostate cancer, prostate infections, and chronic use of certain medications (e.g. ephedrine, pseudoephedrine, phenylpropanolamine, antihistamines such as diphenhydramine, chlorpheniramine etc.) that cause urinary retention especially in men with prostate enlargement.

Despite extensive efforts in both the medical device and pharmacotherapeutic fields, current treatments for BPH remain only partially effective and are burdened with significant side effects. Thus, there remains a need for the development of new devices, systems and methods for treating BPH as well as other conditions in which one tissue or anatomical structure impinges upon or compresses another tissue or anatomical structure.

### SUMMARY

Disclosed herein are devices, systems, and methods for compressing at least a portion of a prostate gland, thereby alleviating pressure on the prostatic urethra, by deploying one or more anchor assemblies or implants into the targeted prostatic tissue. Successful deployment of the implant(s) may effectively treat BPH, among other conditions, for example those in which retraction or compression of enlarged or inflamed tissue is desired.

Also disclosed herein are visualization systems, assemblies, and components thereof configured to provide a real-time view of a treatment site (e.g., within a prostatic urethra) during a treatment procedure, which may involve delivering one or more implants to a targeted prostate gland or other tissue. One or more of the visualization assemblies or components may be included in, attached to, or otherwise coupled with a disclosed implant delivery device.

Also disclosed herein are irrigation systems, assemblies, and components thereof configured to deliver and withdraw an irrigation fluid to and from a treatment site (e.g., within a prostatic urethra) during a treatment procedure, which may involve delivering one or more implants to a targeted prostate gland. Delivering the irrigation fluid may clear the treatment site by removing blood and tissue debris that would otherwise obstruct a view of the treatment site obtained by a scope assembly, non-limiting examples of which may include one or more of the visualization systems, assemblies, and components described herein. One or more of the irrigation assemblies or components may be included in, attached to, or otherwise coupled with a disclosed implant delivery device. One or more of the irrigation assemblies or components may be included in, attached to, or otherwise coupled with a separate treatment device, e.g., a disclosed implant delivery device or component thereof, e.g., shaft assembly. Embodiments of the irrigation system may include an elongate sheath device configured to direct irrigant into a patient. In combination with a delivery device coupled with the introducer sheath, the components may drive fluid flow into and out of a patient, e.g., directing inflow of fresh irrigant through the sheath device and outflow of used irrigant, blood, and/or debris proximally through a shaft assembly of the delivery device.

Treatment procedures pursuant to which the disclosed embodiments may be utilized are not limited to prostate treatments. Treatments of other anatomical structures or tissues may also utilize the disclosed devices, systems, assemblies, and components thereof.

In prostate-specific embodiments, one or more prostatic implants are each configured to anchor simultaneously to the outer prostatic capsule, and also a urethral side, of the lobe of an enlarged prostate, such as a median or lateral lobe. Each implant may include a distal anchor portion (or capsular tab, “CT”) configured to anchor on the outside of the prostatic capsule. An elongate middle portion, such as a suture, may connect the distal anchor portion to a proximal anchor portion (or urethral endpiece, “UE”) configured to anchor to a urethral side of the lobe. Once the distal anchor portion is implanted, the elongate middle portion may be tensioned and the proximal anchor portion attached thereto. Attachment of the proximal anchor portion may lock the tensioned middle portion in place, compressing the prostatic tissue between the distal and proximal anchors and relieving constriction of the prostatic urethra. Devices disclosed herein provide improved means of irrigating and visualizing the treatment site throughout each of these steps.

Each implant or component thereof may be provided in a separate delivery device or cartridge configured to couple therewith. Where a cartridge containing an implant is used, the cartridge may be loaded into a delivery device, which may then be activated by a user to deploy the implant into the targeted tissue by transferring mechanical energy to the cartridge or internal subassembly. The delivery device, or at least its elongate shaft assembly, may then be then removed from the patient, and the spent cartridge replaced with a new cartridge containing a second implant. The delivery device (or more specifically, the shaft assembly) may then be re-inserted and the deployment process repeated. Multiple implants are often necessary to complete a single procedure, thus necessitating multiple cartridge exchanges and the associated removals/reinsertions of the shaft assembly. In some embodiments, a delivery device may be configured to simultaneously hold and serially deploy multiple implants or be reloaded with multiple implants during a procedure, for example via cartridge exchange, in a manner that does not require the elongate shaft assembly to be removed from the patient between deployments.

Embodiments of the delivery devices include various subassemblies mobilized via one or more actuators or manually accessible structures, e.g., triggers, levers, and/or knobs, etc., the operation of which is coordinated and synchronized to ensure accurate and precise implantation of each implant.

In some embodiments, a system configured to deploy one or more implants to a prostate gland of a patient includes a delivery device comprising a handle assembly attached to an elongate shaft assembly. The delivery device may further include a visualization component configured to provide a real-time view of a treatment site inside a patient. The delivery device may further include one or more structures configured to span across an exit port defined by a distal portion of the elongate shaft assembly. The system may further include an elongate sheath device defining an inner lumen configured to receive the elongate shaft assembly, the elongate sheath device configured to direct an irrigant to the treatment site.

In some embodiments, at least a portion of the visualization component may be coupled with the elongate shaft assembly. In some embodiments, the visualization component may include a scope assembly configured to view the distal portion of the elongate shaft assembly at the treatment site. In some embodiments, the scope assembly may include an endoscope housed within a scope tube. In some embodiments, a distal end of the endoscope is positioned proximal to the exit port. In some embodiments, the system further includes an elongate needle configured to extend through the exit port. In some embodiments, the one or more structures comprise rail members. In some embodiments, the rail members are configured to prevent prostatic tissue from obstructing a view of the exit port. In some embodiments, the rail members comprise two rail members defining a space therebetween. In some embodiments, the treatment site comprises a prostatic urethra. In some embodiments, the elongate shaft assembly is configured to accommodate outflow of blood and debris from the treatment site. In some embodiments, the elongate sheath device includes a proximal hub. In some embodiments, the proximal hub includes two ports configured to receive the irrigant from a fluid source. In some embodiments, the proximal hub includes two or more stopcocks configured to control irrigant inflow from a fluid source. In some embodiments, the elongate sheath device includes a connector portion configured to couple a proximal end of the elongate sheath device to the handle assembly. In some embodiments, the connector portion comprises a threaded surface. In some embodiments, the handle assembly includes a locking mechanism configured to engage the connector portion to lock the delivery device to the elongate sheath device. In some embodiments, the elongate sheath device includes an elongate shaft portion, and wherein a cross-sectional gap between an outer surface of the elongate shaft assembly and an inner surface of the elongate shaft portion defines a fluid inflow channel. In some embodiments, the delivery device is configured to enable continuous passive outflow of contaminated irrigant, blood, and/or debris. In some embodiments, the handle assembly includes a fluid outflow channel in fluid communication with the elongate shaft assembly.

These and other examples and objects of the present devices and related methods will be set forth in the following Detailed Description. This Overview is intended to provide non-limiting examples of the present subject matter. The Detailed Description below is included to provide further information about the present devices and related methods. Neither is intended to provide an exclusive or exhaustive explanation of the present devices and methods because this disclosure is written for those of ordinary skill in the art.

### BRIEF DESCRIPTION OF THE DRAWINGS

In the drawings, like numerals can be used to describe similar features and components throughout the several views. The drawings illustrate generally, by way of example but not by way of limitation, various embodiments discussed in the present patent document.

FIG. 1A illustrates a cross-sectional view of the anatomy surrounding a prostate in a human subject.

FIG. 1B illustrates an enlarged cross-sectional view of the anatomy surrounding a prostate.

FIG. 2 shows a coronal section through the lower abdomen of a male human suffering from BPH showing a hypertrophied prostate gland treated with an embodiment of the device of the present disclosure.

FIGS. 3A-3G show the various steps of an example method of treating a prostate gland using the implant(s) and devices shown and described herein.

FIG. 4 shows a perspective view of an example of an implant in accordance with embodiments disclosed herein, for example as shown in FIGS. 2-3G.

FIG. 5 shows a view of an example of an implant delivery device in accordance with embodiments disclosed herein.

FIG. 6 shows a view of another example of an implant delivery device in accordance with embodiments disclosed herein.

FIG. 7 shows a view of a distal end portion of an elongate portion of an implant delivery device in accordance with embodiments disclosed herein.

FIG. 8 shows a view of a distal portion of an implant delivery device in accordance with embodiments disclosed herein.

FIG. 9 shows a view of a distal portion of the implant delivery device shown in FIG. 8 inserted within a urethra during needle insertion into a prostatic lobe in accordance with embodiments disclosed herein.

FIG. 10 shows a view of a distal portion of the implant delivery device shown in FIG. 8 inserted within a urethra, and a distal portion of another implant delivery device inserted within a urethra.

FIG. 11 shows a cross-sectional perspective view of a portion of a shaft assembly in accordance with embodiments disclosed herein.

FIG. 12 shows a partially transparent view of a portion of a delivery device in accordance with embodiments disclosed herein.

FIG. 13 shows another view of the portion of the delivery device shown in FIG. 12 in accordance with embodiments disclosed herein.

FIG. 14 shows a view of a portion of a delivery device in accordance with embodiments disclosed herein.

FIG. 15 shows another view of the portion of the delivery device shown in FIG. 14 in accordance with embodiments disclosed herein.

FIG. 16 shows another view of a portion of the delivery device shown in FIGS. 14 and 15 in accordance with embodiments disclosed herein.

FIG. 17 shows a view of a portion of a delivery device in accordance with embodiments disclosed herein.

FIG. 18 shows a partially transparent view of the portion of the delivery device shown in FIG. 17 in accordance with embodiments disclosed herein.

FIG. 19 shows another view of the portion of the delivery device shown in FIG. 17 in accordance with embodiments disclosed herein.

FIG. 20 shows another view of the portion of the delivery device shown in FIG. 17 in accordance with embodiments disclosed herein.

FIG. 21 shows a perspective view of a sheath device in accordance with embodiments disclosed herein.

FIG. 22 shows a perspective view of another sheath device in accordance with embodiments disclosed herein.

FIG. 23 shows a perspective view of another sheath device in accordance with embodiments disclosed herein.

FIG. 24 shows a schematic of a side view of a delivery system that includes a sheath device in accordance with embodiments disclosed herein.

FIG. 25 shows a schematic of Detail 12-12 of FIG. 24, showing a magnified view of the distal portion of the delivery system.

FIG. 26 shows views of a sheath device attached to a delivery device in accordance with embodiments disclosed herein.

FIG. 27 shows views of a visual obturator compatible with the delivery devices and sheath devices disclosed herein.

The drawing figures are not necessarily to scale. Certain features and components may be shown exaggerated in scale or in schematic form and some details may not be shown in the interest of clarity and conciseness.

### DETAILED DESCRIPTION

The present devices and associated methods provide clinicians with means to treat an enlarged prostate, which may be a symptom of BPH, to alleviate its impingement on the adjacent prostatic urethra. Implants disclosed herein can be placed using a disclosed device pursuant to a method for compressing a prostate gland or portion thereof according to the following description.

As used herein, the terms “prostatic implant” and “implant” and “anchor assembly” and “retainer” may be used interchangeably. Each implant, once fully assembled, may include a distal anchor or capsular tab (hereinafter “CT”), a connector member, portion or suture (hereinafter “suture”), and a proximal anchor or urethral endpiece (hereinafter “UE”). The CT and suture may be provided together as a unitary component or assembly, with the CT attached, fixed, integrally formed with, or coupled to one end of the suture. The UE may be provided as a separate component that is attached to the CT/suture after deploying the CT/suture to the targeted tissue, for instance after the CT has emerged from the distal end of the delivery needle at or beyond the outer surface of the prostatic capsule, and the suture has been implanted within the prostatic tissue. Embodiments include attaching the UE to the suture of a CT/suture assembly after tightening the CT against the outer capsular surface and tensioning the suture through the targeted tissue.

The terms “actuator” and “actuator member” may be used interchangeably in embodiments disclosed herein. In some examples, an “actuator” or “actuator member” may include or be synonymous with a manually engageable portion, member, part, or component (such as a button, dial, switch, toggle, knob, lever, or trigger) configured to be actuated, for example via manually induced movement (such as a button press or lever sweep).

As used herein, the term “suture” may be used to represent a connector, connector member, connector portion, or elongate middle portion or member of an implant extending between a distal anchor and a proximal anchor, through the prostatic tissue.

The term “member” may be used herein to represent a subcomponent or subassembly of a larger component or assembly, or the term may represent the larger component or assembly itself. The terms “member” and “component” and “element” may be used interchangeably herein.

The terms “elongate member” and “shaft” and “shaft assembly” may refer to the same or similar components and may be used interchangeably herein. Embodiments of a shaft assembly may include components configured to access a treatment site within a prostatic urethra, deploy one or more implants, irrigate the treatment site, and/or visualize the procedure from within the patient. Examples of the shaft assembly may include a scope tube configured to accommodate an endoscopic instrument or scope assembly within a lumen of the scope tube. Examples of a shaft assembly may include one or more components of a needle assembly, a suture assembly, and/or a cutter assembly, non-limiting examples of which are disclosed in U.S. Pat. No. 11,298,115 and US Patent Application Publication No. 2021/0378658, the entire contents of each of which are incorporated by reference herein.

The term “sheath” may be used interchangeably with “sheath device” and/or “introducer sheath.” Embodiments of the sheaths disclosed herein can be configured to receive an elongate portion or member, e.g., shaft assembly, of the delivery devices disclosed herein.

The term “procedure” may refer to a medical treatment method used to compress at least a portion of an anatomical structure or tissue, including tissue of the prostate gland, which may be enlarged relative to a corresponding healthy tissue.

The term “user” may refer to a clinician, doctor, nurse, or medical professional performing a procedure described herein, which may involve the implantation of one or more implants within a targeted portion of prostatic tissue, which may be enlarged relative to normal prostatic tissue. In some examples, the term “user” may refer to more than one person, including two or more medical professionals working together to perform a procedure.

The terms “deploy” and “implant” and “deliver” may be used interchangeably herein, referring to the release and/or ejection of a disclosed implant or subassembly from a delivery device into a tissue being targeted. Accordingly, for instance, an implant may be fully deployed, delivered, or implanted when the distal anchor of an implant is positioned at an outer surface of the prostatic capsule, the suture has been advanced through and tensioned within the targeted prostate lobe, and the UE has been attached to the tensioned suture at the urethral side of the lobe.

The terms “distal anchor” and “capsular tab” and “CT” may be used interchangeably herein. The terms “proximal anchor” and “urethral endpiece” and “UE” may be used interchangeably herein.

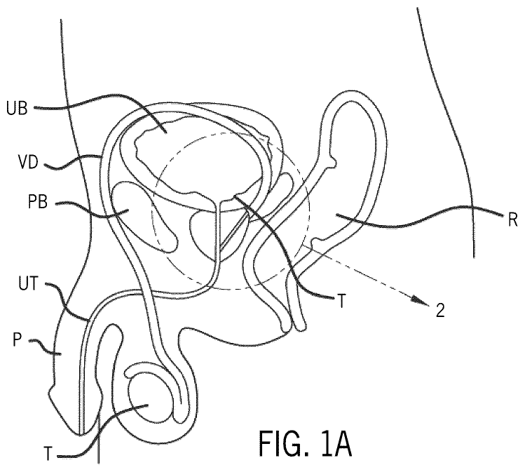

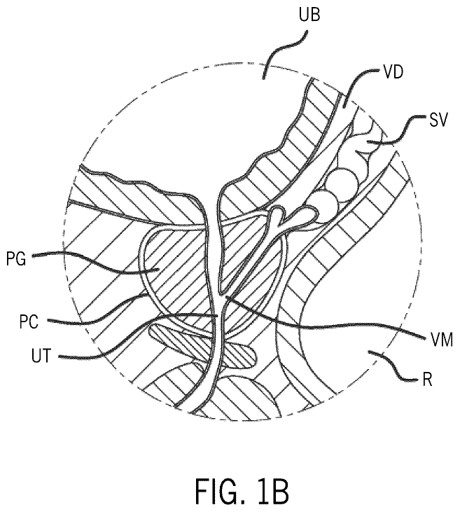

FIGS. 1A and 1B illustrate various features of the urological anatomy of a human subject. The prostate gland PG is a walnut-sized muscular gland located adjacent the urinary bladder UB. The urethra UT runs through the prostate gland PG. The prostate gland PG secretes fluid that protects and nourishes sperm. The prostate also contracts during sperm ejaculation to expel semen and provide a valve to keep urine out of the semen. A firm prostatic capsule PC surrounds the prostate gland PG.

The urinary bladder UB holds urine. The vas deferentia VD define ducts through which semen is carried, and the seminal vesicles SV secrete seminal fluid. The rectum R is the end segment of the large intestine through which waste is dispelled. The urethra UT carries both urine and semen out of the body. Thus, the urethra is connected to the urinary bladder UB and provides a passageway to the vas deferentia VD and seminal vesicles SV.

The trigone T is a smooth triangular end of the bladder. It is sensitive to expansion and signals the brain when the urinary bladder UB is full. The verumontanum VM is a crest in the wall of the urethra UT where the seminal ducts enter. The prostatic urethra is the section of the urethra UT that extends through the prostate.

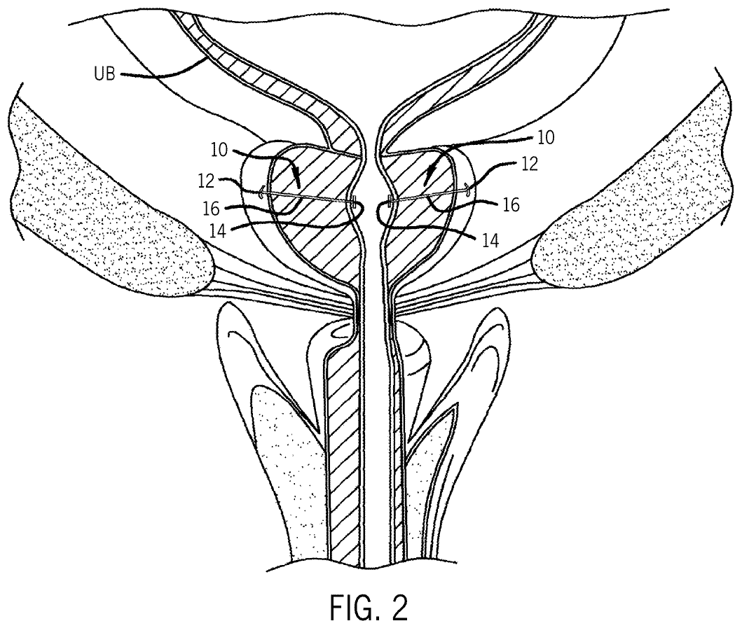

FIG. 2 shows a coronal section through the lower abdomen of a male human suffering from BPH showing a hypertrophied prostate gland treated with an embodiment of the device of the present invention. It has been discovered that the enlarged prostate gland is compressible and can be retracted so as to relieve the pressure from the urethra. In accordance with one embodiment of the present invention, a retaining device can be placed through the prostate gland in order to relieve the pressure on the urethra. In FIG. 2, a retainer or implant 10 is implanted in the prostate gland. Implant 10 comprises a distal anchor or capsular tab 12 (or “CT”) and a proximal anchor or urethra endpiece (or “UE”) 14. After assembly of the implant 10, the distal anchor 12 and proximal anchor 14 are connected by a middle portion or connector 16, which may comprise, include, or resemble a suture. The radial distance from the urethra to distal anchor 12 is greater than the radial distance from the urethra to the proximal anchor 14. The distance or tension between the anchors is sufficient to compress, displace or change the orientation of an anatomical region between distal anchor 12 and proximal anchor 14. The connector 16 may be substantially inelastic so as to maintain a constant force or distance between the proximal and distal anchors, or it may be elastic to facilitate drawing the proximal and distal anchors closer together. In the embodiment shown in FIG. 2, distal anchor 12 is located on the outer surface of the capsule of prostate gland CP and acts as a capsular anchor. Alternatively, distal anchor 12 may be embedded inside the tissue of prostate gland PG, or in the surrounding structures around the prostate, such as periosteum of the pelvic bones, within the bones themselves, pelvic fascia, muscles traversing the pelvis or bladder wall. Also, in the embodiment shown in FIG. 2, proximal anchor 14 may be located on the inner wall of urethra UT, where it acts as a urethral anchor. Alternatively, proximal anchor 14 may be embedded inside the tissue of prostate gland PG or surrounding structures as outlined above. Distal anchor 12 and proximal anchor 14 are implanted in the anatomy such that a desired distance or tension is created in connector 16. This causes distal anchor 12 and proximal anchor 14 to retract or compress a region of prostate gland PG to relieve the urethral constriction. In FIG. 2, two implants 10 are implanted in a lateral lobe (side lobe) of prostate gland PG. The various methods and devices disclosed herein may be used to treat a single lobe or multiple lobes, including one or more lateral lobes and the median lobe, of the prostate gland or other anatomical structures, by deploying one or more implants to the targeted tissue.

The implants may be deployed at particular angles relative to the axis of the urethra to target one or more lateral lobes and/or median lobe of the prostate gland. For example, implant 10 may be deployed between the 1 o'clock and 3 o'clock position relative to the axis of the urethra to target the left lateral lobe of the prostate gland. In another example, implant 10 may be deployed between the 9 o'clock and 11 o'clock position relative to the axis of the urethra to target the right lateral lobe of the prostate gland. In another example, implant 10 may be deployed between the 4 o'clock and 8 o'clock position relative to the axis of the urethra to target the middle lobe of the prostate gland.

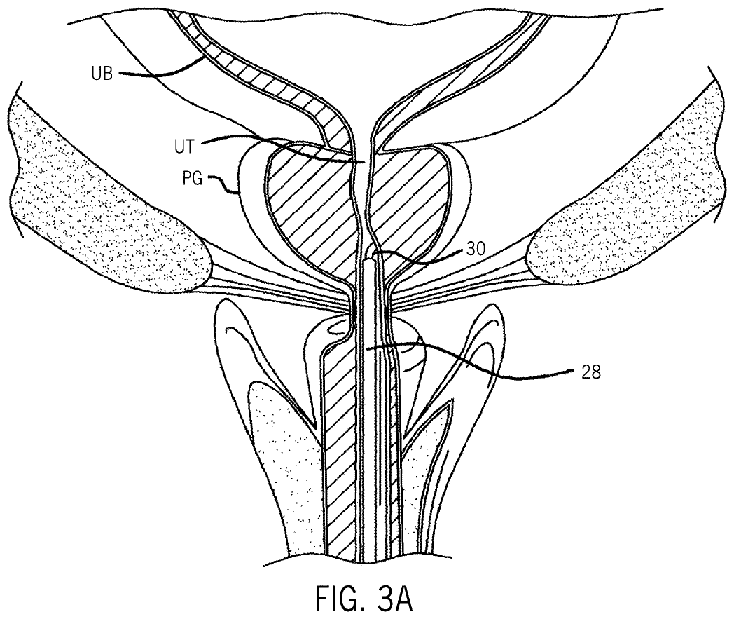

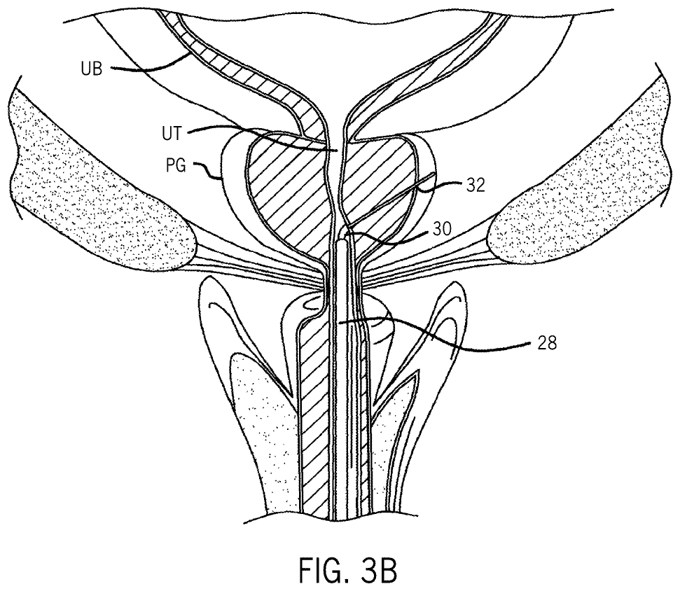

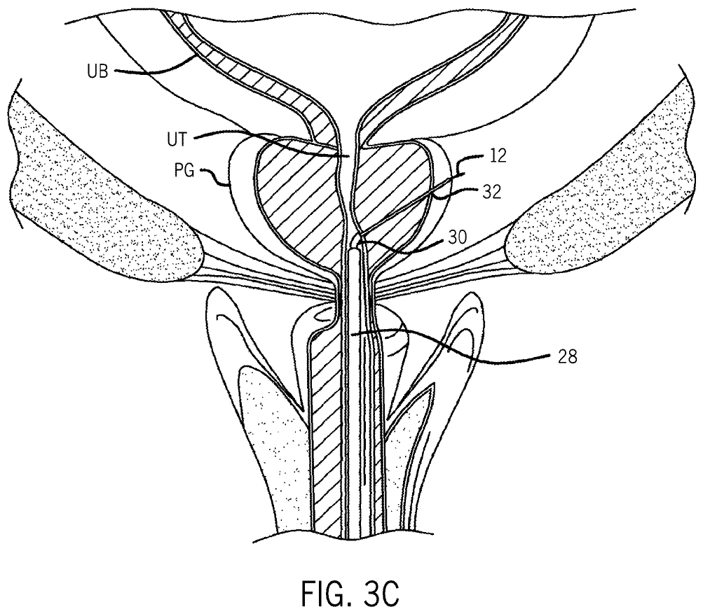

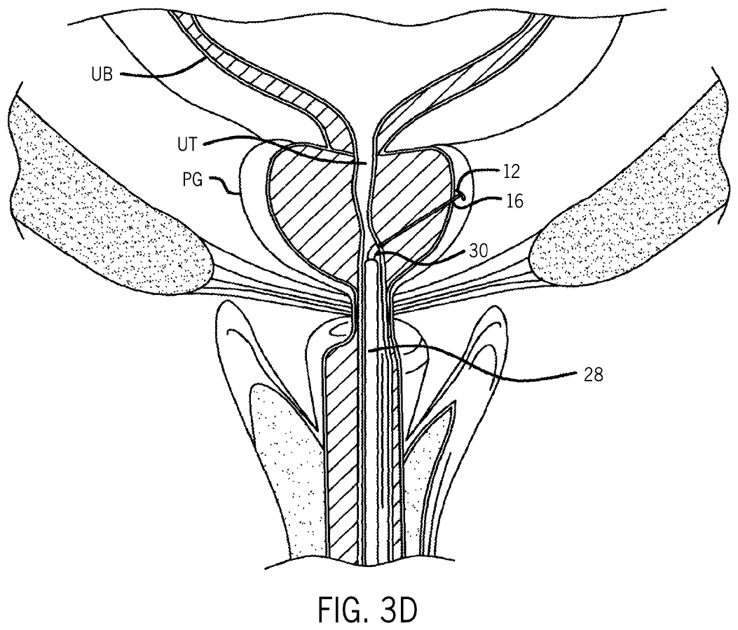

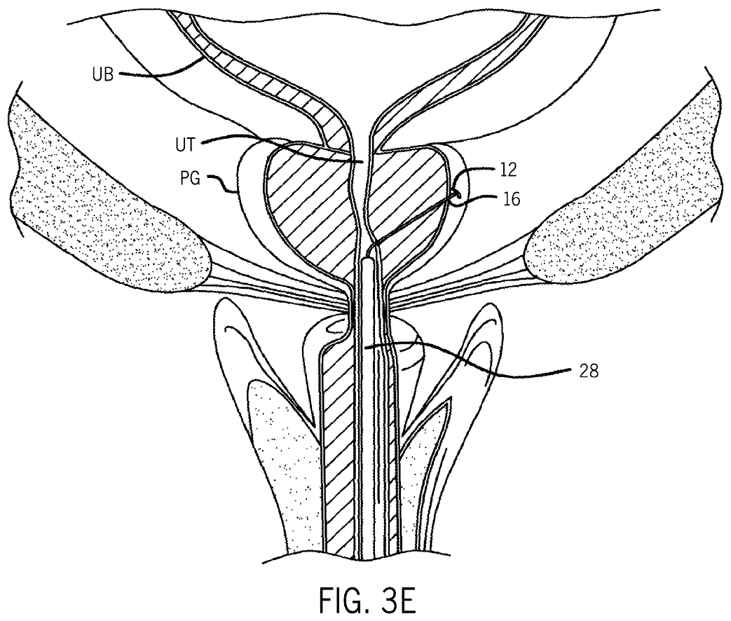

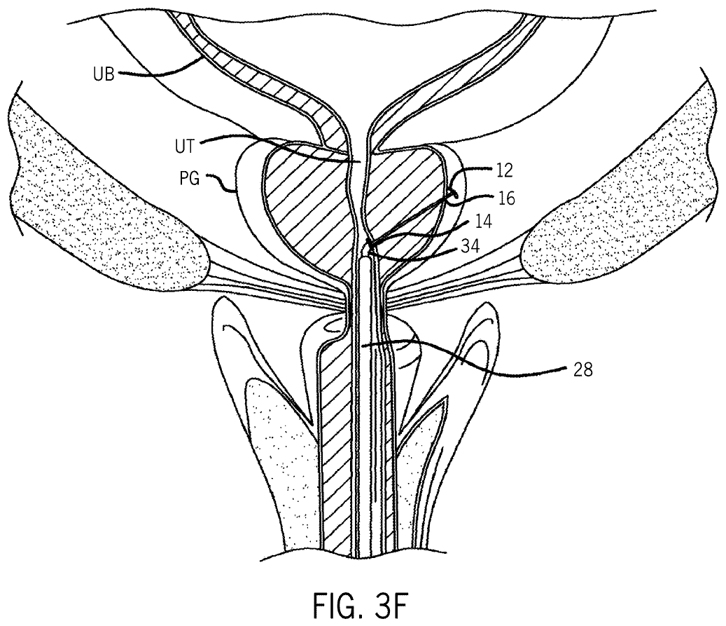

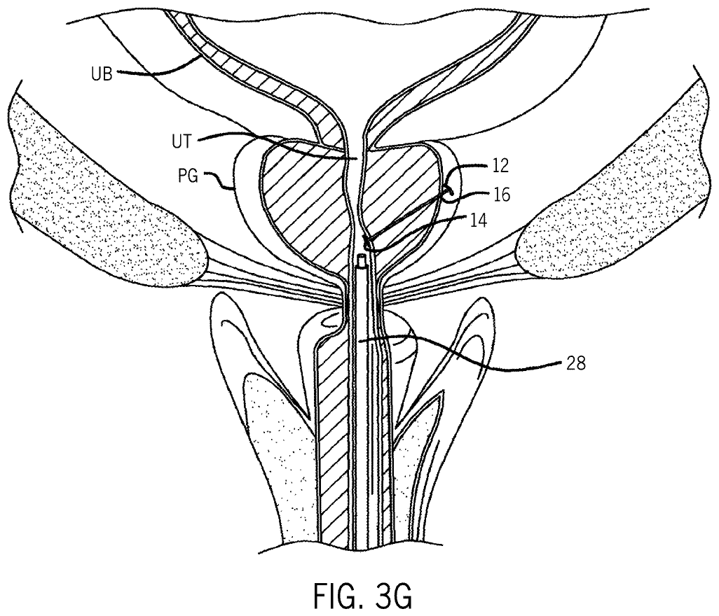

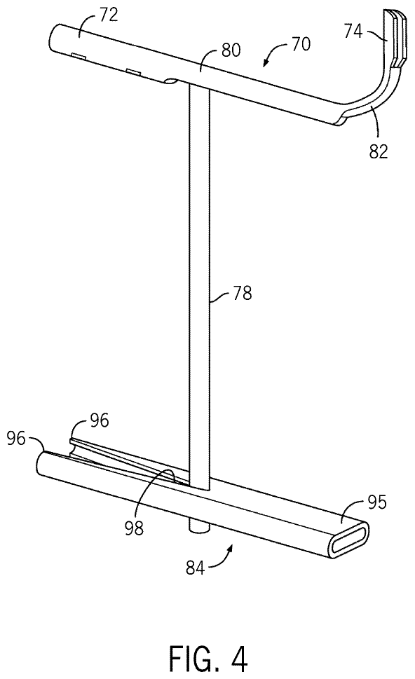

FIGS. 3A-3G show the various steps of a non-limiting example of a method of treating a prostate gland using one or more of the implants and associated delivery devices disclosed herein (see e.g., FIG. 4). One or more of the illustrated steps may be modified or excluded in accordance with certain embodiments disclosed herein, for example those not requiring a sheath. One or more of the depicted steps, shown for illustrative purposes, may be performed in a different manner depending on the particular delivery device(s) used, embodiments of which are disclosed herein. Similar methods may also be implemented to deploy retainers, implants, or compression devices in other anatomical structures.

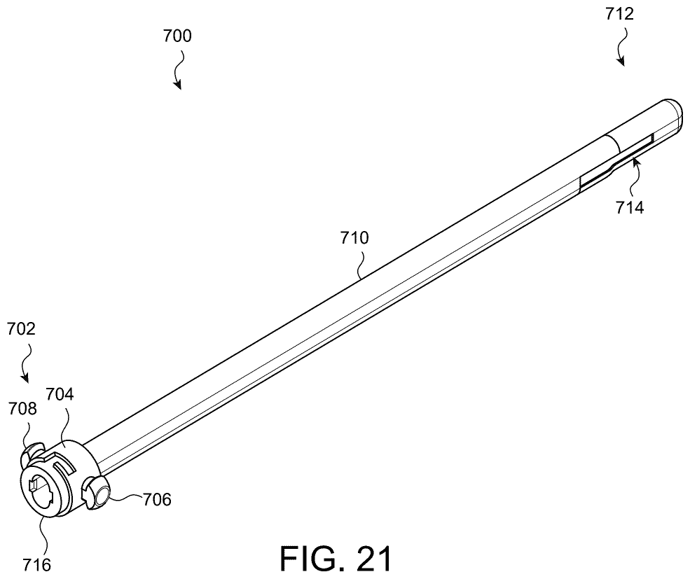

In the step shown in FIG. 3A, a sheath 28, which may comprise a standard resectoscope sheath or the sheath shown in any of FIGS. 21-26, may be introduced into the urethra (trans-urethrally). The sheath 28 may be advanced through urethra UT such that the distal end of sheath 28 is positioned near a region of urethra UT that is obstructed by a hypertrophied prostate gland PG. A distal anchor delivery device 30 featuring an elongate member or shaft may then be introduced through sheath 28. Distal anchor delivery device 30 may be placed in the sheath 28 after the distal end of the sheath 28 is positioned near the region of the urethra UT that is obstructed, or the distal anchor delivery device 30 may be pre-loaded in the sheath 28 before positioning of the sheath 28. Distal anchor delivery device 30 may be advanced through sheath 28 such that the distal end of distal anchor delivery device 30 emerges out of the distal end of sheath 28. Distal anchor delivery device 30 may be oriented such that a working channel opening of distal anchor delivery device 30 points towards a lateral lobe of prostate gland PG. During the procedure, the sheath 28 may be used to irrigate the treatment site, for example within the prostatic urethra. Irrigation performed by the sheath 28 may include both the injection and withdrawal of inflation fluid to and from the treatment site.

In the step shown in FIG. 3B, a penetrating member or needle 32 may be introduced through distal anchor delivery device 30. Needle 32 may be placed in distal anchor delivery device after the distal anchor delivery device 30 is advanced through sheath 28, or the needle 32 can be pre-loaded in the distal anchor delivery device 30. In one non-limiting embodiment, the needle 32 is a 20-gauge needle. The needle may comprise nitinol in some embodiments. Needle 32 is advanced through distal anchor delivery device 30 such that it emerges through the working channel opening. Needle 32 may be further advanced until it penetrates through the tissue of prostate gland PG and the distal end of needle 32 emerges out of the capsule of prostate gland CP.

In the step shown in FIG. 3C, the distal anchor 12 connected to the connector 16 may be deployed at, or just beyond, the outer surface of the prostatic capsule. In some embodiments, the distal anchor 12 may be deployed by being extended beyond the distal end of the needle 32. In other embodiments, the distal anchor 12 may be deployed by being held in place by a pusher or connector while the needle 32 is retracted, thus exposing the distal anchor and unsheathing distal anchor 12 and connector 16 upon continued needle retraction. Distal anchor 12 can be pre-loaded within the needle 32 or it can be loaded in the needle 32 after the needle 32 has been advanced through distal anchor delivery device 30.

In the step shown in FIG. 3D, the needle 32 may be removed from the distal anchor delivery device 30 by pulling the needle 32 in the proximal direction. In the step shown in FIG. 3E, the distal anchor delivery device 30 may be removed from the sheath 28 by pulling distal anchor delivery device 30 in the proximal direction. Also, the connector 16 may be pulled to orient the distal anchor 12 approximately perpendicularly or otherwise transverse to the connector 16, against the outer surface of the prostatic capsule.

In the step shown in FIG. 3F, the connector 16 may be passed through the proximal anchor 14 located on a proximal anchor delivery device 34. The proximal anchor delivery device 34 may be advanced through the sheath 28 such that the distal end of proximal anchor delivery device 34 emerges out of the distal end of sheath 28. A desired tension may be introduced in connector 16 such that distal anchor 12 is pulled by connector 16 with a desired force. The proximal anchor can additionally or alternatively be visualized through an endoscope or under fluoroscopy and advanced along the connector 16 until the desired retraction of the tissue is achieved. In other embodiments, the proximal anchor may be a v-shaped or clothespin-shaped piece that may be forced, in some cases at high speed, onto the connector 16 to fixedly engage the connector.

In the step shown in FIG. 3G, the connector 16 is attached to proximal anchor 14. Proximal anchor 14 is also released from proximal anchor delivery device 34, thus deploying proximal anchor 14 and the implant 10 as a whole in the targeted anatomy. Proximal anchor delivery device 34 and sheath 28 may then be removed from the anatomy, retracting proximally through the urethra.

This method may be used to retract, lift, support, reposition or compress multiple regions or lobes of the prostate gland PG. In the method shown in FIGS. 3A-3G, the distal anchor 12 is deployed on the outer surface of the prostatic capsule. Thus, the distal anchor 12 acts as a capsular anchor. Alternatively, distal anchor 12 may be deployed inside the tissue of prostate gland PG or beyond the prostate as outlined previously. Similarly, in the method shown in FIGS. 3A-3G, the proximal anchor 14 may be deployed on the inner wall of urethra UT, acting as a urethral anchor. Alternatively, proximal anchor 14 may be deployed inside the tissue of prostate gland PG. In some examples, as noted above, one or more of the delivery components or devices may not be used or necessary, non-limiting examples of which may include sheath 28.

Treatment procedures may involve repeating one or more, including all, of the steps shown in FIGS. 3A-3G to deliver multiple implants to the targeted tissue. One or more steps may also be modified, for example pursuant to approaches utilizing a delivery device pre-loaded with two or more implants that may be deployed in serial fashion without removing the elongate member or shaft assembly of the delivery device from the urethra. Constant irrigation and visualization of the prostatic urethra may be especially imperative when using such “multi-fire” delivery devices.

FIG. 4 provides a perspective view of a non-limiting example of an anchor assembly or implant deployed using the systems, devices, and assemblies herein. In an unconstrained configuration, the distal anchor component 70 (or “CT”) may include a head portion 72 which may be generally orthogonally oriented with respect to a tail portion 74. While housed in the distal portion of a delivery needle and prior to deployment at a target area, the distal anchor component 70 may be constrained to a generally straight configuration, only subsequently assuming the unconstrained (i.e., orthogonally oriented) configuration upon deployment from the needle assembly.

In certain embodiments, the distal anchor component 70 may be formed from a nitinol base stock that is generally tubular and can be shape-set to include the orthogonally oriented configuration of the head portion 72 with respect to the tail portion 74. A suture 78 may be attached to the distal anchor component 70. In one embodiment, a polyethylene terephthalate (PET) suture portion 78 is thermoformed onto locking features in the distal anchor component 70. The distal anchor component 70 may be locally heated to re-flow the suture onto the end of the distal anchor component 70 and into cutouts on the distal anchor component 70. The distal anchor component 70 may be attached to the suture portion 78 through any of several known techniques for bonding a PET material to a nitinol material.

In one embodiment, a mid-section 80 of the distal anchor component 70 provides a structural transition from the head portion 72 to the tail portion 74 and has a portion of a side wall removed in the area of mid-section 80. A further portion of the side wall is removed to define a connector section 82 of the tail portion 74 which extends from the mid-section 80. In one embodiment, this connector section 82 may include a bend that creates the orthogonally oriented configuration. Thus, in its pre-implanted form, the anchor assembly can include a distal anchor component 70 whose initial engagement with a suture portion 78 is generally coaxial.

Still referring to FIG. 4, in one embodiment the proximal anchor component 84 (or “UE”) includes prongs 96 that grip the suture portion 78. The interior structure of the prongs 96 may function to disrupt the surface of the suture portion 78, both pressing into the suture portion 78 and compressing the suture portion 78 therebetween. A tab 98 may be included, extending from one or more of the prongs 96 to help create secure engagement between the proximal anchor component 84 and the suture portion 78. In some examples, the proximal anchor component 84 may comprise stainless steel. In some embodiments, the implant in its entirety may comprise a polymer composition, for example resembling a “T” in whole or in part.

In certain embodiments, the proximal anchor component 84 may be present in the shaft assembly of a delivery device in a configuration that is separate and disconnected from the distal anchor component 70 and the suture portion 78, which may be engaged with each other and contained within the needle assembly. After the distal anchor component 70 and the suture portion 78 have been placed within tissue, the proximal anchor component 84 may be securely engaged with the suture portion 78 to form the fully assembled anchor assembly or implant. To facilitate engagement of the proximal anchor component 84 with the suture portion 78, the proximal anchor component 84 may include, in some examples, a rigid, generally cylindrical back end 95. This rigid, generally cylindrical back end 95 can be used to push the proximal anchor component 84 into engagement with the suture 78 via transfer of the mechanical energy in the handle of the associated delivery device.

The tissue approximation anchors disclosed herein and shown in FIG. 4 may be designed to be useable in a physician's clinical office environment (in contrast to requiring a hospital environment) with a delivery tool. The delivery tool may be used with a 19 F or 20 F sheath in some examples. Additionally, the material selection and construction of the tissue approximation anchor still allows for a subsequent TURP procedure to be performed, if necessary, on the prostate. In this suture-based, tissue approximation technique, a needle delivery mechanism may be used to implant an anchor assembly.

The implants disclosed herein may be delivered to a targeted lobe of a prostate gland using a delivery system that further includes a delivery device comprising a tubular elongate member (or shaft assembly) and at least one hollow delivery needle configured to be advanced therethrough. The needle may have a sharp distal tip configured to pierce the prostate gland, including the outer capsule, along with an inner lumen configured to house the distal anchor component (or CT) and the suture (or other middle portion or connector of an implant), ready for deployment. The delivery system may also include a sheath into which the elongate shaft assembly of the delivery device is inserted. In operation, the sheath can be inserted into the urethra of a subject before insertion of the shaft assembly. Accordingly, the cross-sectional diameter of the sheath may be greater than that of the shaft assembly.

Examples of a delivery device may generally include a handle assembly supporting an elongate portion comprising a tubular elongate member in the form of or comprising a shaft assembly. The elongate member may be substantially rigid or flexible and defines a cross-sectional diameter that is approximately equal to or less than that of the urethra, i.e., it may have low profile suited to navigate body anatomy, e.g., urethra, to reach an interventional site, e.g., prostatic urethra. An elongate shaft member or assembly may include one or more lumens. Substructure may be provided to maintain a longitudinal profile of the elongate member so that the interventional procedure can progress as intended. Embodiments of the delivery device may also include an endoscope, providing the ability to view the interventional procedure, which may be positioned within an elongate scope tube within the elongate member. The elongate member may be sized to fit within a cystoscopic sheath for patient tolerance during a procedure in which the subject is awake rather than under general anesthesia. Non-limiting examples of the sheath may be 19 F or 20 F. Using the disclosed systems, insertion of one or more implants in a prostate gland may be performed in an outpatient setting.

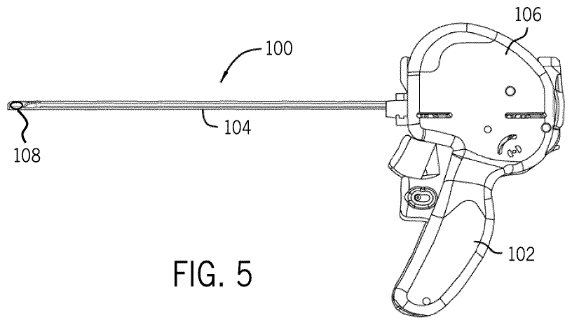

FIG. 5 illustrates one non-limiting example of a delivery device 100 having structure configured to gain access to an interventional site and deploy a prostatic implant, such as implant 10. As shown, the delivery device 100 may include a handle assembly 102 connected to a tubular elongate member 104 (or shaft assembly), which may surround an elongate delivery needle defining an inner lumen and arranged coaxially to the elongate member 104.

The delivery device 100 may further include a number of subassemblies configured to deliver and employ an implant at a target site. A handle case assembly 106, including handle parts that form part of the handle assembly 102, is also included. The handle assembly 102 is sized and shaped to fit comfortably within an operator's hand and can be formed from conventional materials. Windows can be formed in the handle case assembly 106 to provide access to internal mechanisms of the device so that a manual override is available to the operator in the event the interventional procedure needs to be abandoned.

In some examples, the elongate member 104 (or shaft assembly) may define at least one inner lumen sized and configured to accommodate longitudinal insertion of at least the hollow delivery needle and prostatic implant therethrough, with the distal anchor member or CT of the implant nestled within the delivery needle, and the proximal anchor component or UE included in the elongate member 104 in a delivery configuration. In some examples, the CT and middle portion or suture may be enclosed within a first lumen of the elongate member 104, and the UE may be enclosed within a second lumen of the elongate member 104. The elongate member 104 may have a shape and/or flexibility configuring it to navigate through a urethra without kinking or puncturing the urethral wall. In some examples, the elongate member 104 may be substantially rigid, such that it maintains an approximately straight configuration during its insertion through the urethra. According to such examples, the distal portion of the elongate member 104 may be angled toward or away from various anatomical features surrounding the urethra, e.g., one or more lobes of the prostate gland, by adjusting the angular orientation of the proximal end of the elongate member 104 outside the body. The distal end of the elongate member 104 may comprise smooth, blunt, and/or beveled surfaces to avoid puncturing the urethral wall. Embodiments may include a sidewall opening 108 (or exit port) defined by a distal portion of the elongate member 104. The sidewall opening 108 may be sized and configured to accommodate passage of the hollow delivery needle therethrough.

In some examples, the handle assembly 102 may simultaneously contain two or more implants, e.g., two, three, four, five, six, seven, eight, nine, ten implants, or more. Such embodiments may include internal subassemblies configured to deploy the implants in serial fashion.

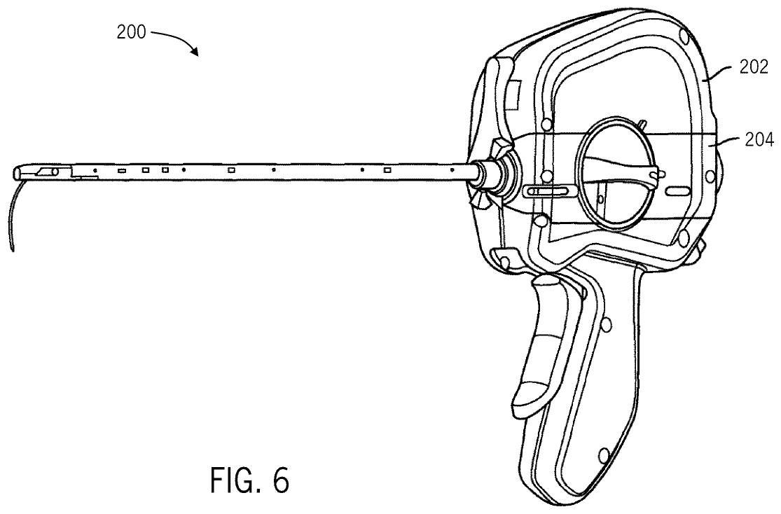

In some examples, a delivery device may include a handle assembly and a removable cartridge configured to couple therewith. For instance, FIG. 6 illustrates a non-limiting example of a delivery device 200 that includes a handle assembly 202 and complementary cartridge 204 that can be used to deliver one or more implants to prostatic tissue, for example pursuant to a method of treating benign prostatic hyperplasia. The handle assembly 202 includes sources of mechanical energy that may be transferred to the cartridge 204 to deploy the anchor assembly within the cartridge 204. The handle assembly may be configured such that the energy can be restored to these mechanical energy sources while the first cartridge is being used and/or prior to the insertion of a second cartridge. The handle assembly 202 may be designed to reliably deliver energy to multiple cartridges in sequence before a new handle assembly is required. In some examples, each cartridge 204 may contain one implant or portion thereof, e.g., one CT/suture assembly. The handle assembly 202 may contain one or more components or portions of an implant, e.g., one or more urethral endpieces. In some examples, removal of the cartridge 204 requires retraction of the elongate shaft assembly from a patient. In other examples, removal of the cartridge 204 may not require retraction of the elongate shaft assembly. The configuration of the cartridge 204 may vary.

Embodiments of the delivery device may include a variety of additional or substitute components and subassemblies, such as those described in U.S. Pat. Nos. 10,130,353, 11,298,115, and U.S. Patent Application Publication No. 2021/0378658, the entire contents of each of which are incorporated by reference herein.

Forming one or more of the devices, device components, and device assemblies disclosed herein may involve one or more molding processes, metal-working, and/or multi-part assembly. Plastic components of the various devices can be injection molded in some embodiments. Plastic components may be extruded in some examples, and may undergo secondary processing, such as thermal tipping and/or over-molding. Metal components may be formed via stamping processes. Laser welding may be employed to fix metal components to each other. A variety of connector components and tools, e.g., snaps, screws, etc., may be used to assemble a given device.

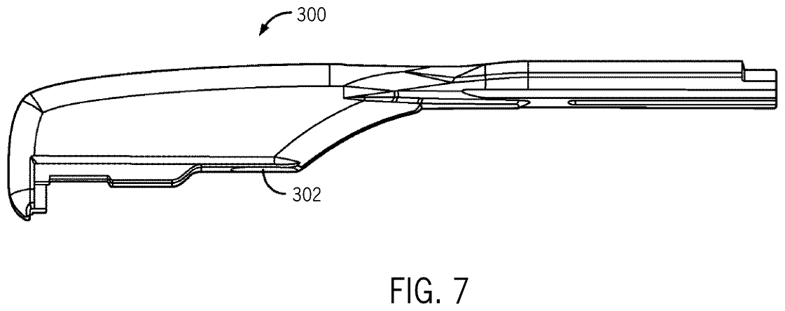

FIG. 7 provides a side view of a portion of a distal tip of a portion of an elongate shaft assembly, which may constitute or be coupled with a handle assembly or a cartridge in various embodiments. As shown, the shaft distal portion 300 may include a shaft distal portion exit port 302 from which a distal portion of a delivery needle (not shown) emerges when the needle distal portion is extended from the shaft distal portion 300. The exit port 302 may therefore constitute the distal terminus of an elongate shaft lumen, which may define an effective radius of curvature as the lumen transitions from running along the long axis of the shaft assembly to running in a direction transverse to the long axis of the shaft assembly in some examples. Additional, non-limiting features of an example of a shaft assembly or distal portion thereof are described in U.S. Pat. No. 11,298,115.

At or near the exit port 302, a UE may be attached to each of one or more sutures implanted and tensioned through a targeted lobe of a prostate gland. After or during the UE attachment process, the excess suture proximal to the attached UE may be cut. Visualizing one or more steps involved in the implant deployment process, including navigation of the shaft assembly to a treatment site, positioning of the distal end of the shaft assembly adjacent to a targeted lobe, insertion of the distal tip of a delivery needle containing one or more implant components into the targeted tissue, tightening of the suture, and/or placement of the UE, among other steps, may be imperative to completing the treatment procedure effectively, which may involve the deployment of multiple implants.

Embodiments of one or more implant delivery systems disclosed herein may include one or more visualization components, assemblies, or systems configured to enable real-time viewing of a procedure at or near a treatment site, for instance within or near a prostatic urethra. In some examples, one or more visualization components can be included, integrated, or otherwise coupled with one or more components of the implant delivery device, such as within at least a portion of the elongate shaft assembly, where the component(s) can provide a real-time view of the procedure from within the patient. Examples may provide a view of the needle exiting the device and advancing into the targeted prostatic lobe. Examples may also be configured to provide a real-time view of the suture tensioning and/or UE attachment process.

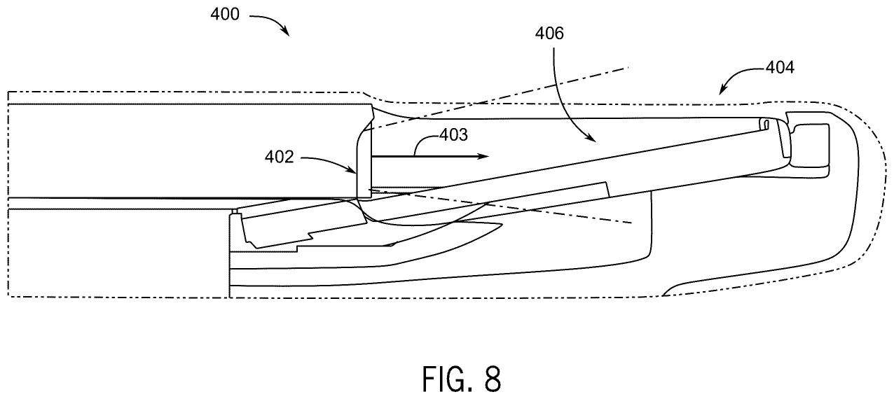

One non-limiting embodiment of a portion of a delivery device configured to provide real-time visualization of an implant procedure is shown in FIG. 8. As shown, a distal end portion of a shaft assembly 400 of a delivery device, which may also include a needle assembly in some examples, may include or be configured to receive or contain a distal end of a camera or viewing device, non-limiting examples of which may include a cystoscope or endoscope (hereinafter “scope 402”). The scope 402 may be provided within or advanced through a separate scope lumen extending through the shaft assembly in some examples.

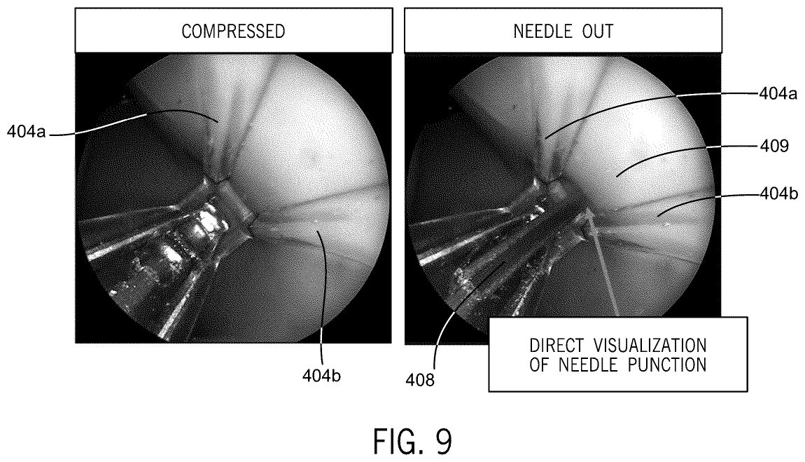

One or more elongate structures, members, portions or segments, e.g., rail members or guard rails 404, included in the shaft assembly may span across an exit port 406 through which a needle containing a CT/suture assembly may be extended during a procedure. The rail members or guard rails 404 (used interchangeably herein) may be rigid or substantially rigid such that they maintain their shape even when surrounded by enlarged, inflamed, or otherwise encroaching tissue, which may may otherwise extend into the exit portion 406. The scope 402 may be positioned or mounted above or across from the needle and UE (both of which may be advanced through the exit port 406 during a procedure), relative to the longitudinal axis of the shaft assembly, where the scope 402 may provide a direct view of the needle as it punctures the targeted prostatic lobe. As further shown in FIG. 9, guard rails 404a, 404b may be configured to retain or otherwise hold back a portion of the adjacent prostatic tissue during the procedure to maintain an unobstructed view of the treatment site during compression. The space between the guard rails 404a, 404b may be configured to accommodate passage of a needle 408 therethrough. The needle 408 may extend laterally outward, for example along a curved trajectory relative to the longitudinal axis of the shaft assembly, toward and into the target tissue 409. The scope 402 may provide a direct view of the prostatic urethra and needle 408 being advanced/retracted through the exit port 406, such that the site of needle puncture can be directly visualized (see arrow 403), thereby enabling more accurate tissue targeting. The configuration shown in FIGS. 8 and 9 may also eliminate the need for one or more ancillary visualization components and exchange of the same, which may improve patient comfort and simplify the device and procedure as a whole.

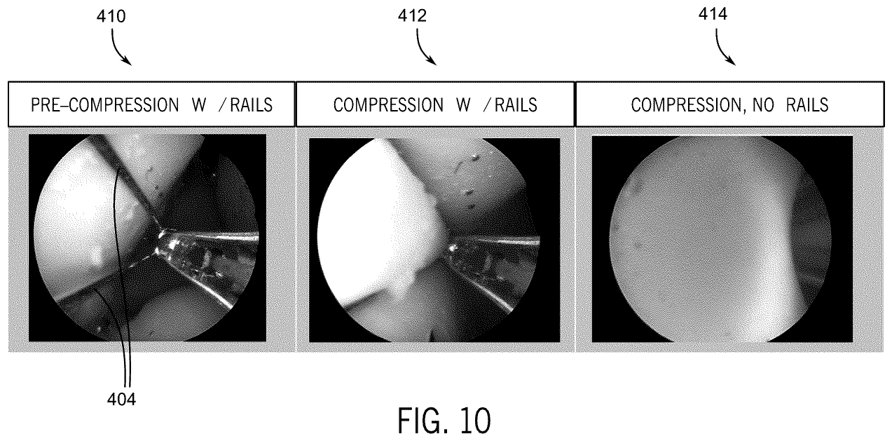

FIG. 10 provides photographic views of a treatment site during a procedure in which the shaft assembly 400 illustrated in FIG. 8 is utilized, versus a procedure in which a different shaft assembly lacking the guard rails 404 is used. Image 410 shows the treatment site prior to tissue compression, for reference, and images 412 and 414 show the same treatment site after compressing the tissue using a device with guard rails 404 and without guard rails, respectively. As shown, the view of the treatment site remains substantially unobstructed when the guard rails 404 are incorporated into the device. By contrast, image 414 shows that the view of the treatment site may be substantially obstructed when the guard rails are absent.

The configuration of the visualization system may vary. The guard rails 404, for instance, may be included in a variety of shapes, sizes, and positions. Embodiments may include two elongate shafts, rails, or bars, as shown in FIGS. 9 and 10. Examples may include two or more rails, e.g., three, four, five, six rails or more, or fewer, i.e., only one rail. Examples may additionally or alternatively include one or more cross-bars connecting two rail members. Such embodiments may include one cross-bar or two, three, four, five, six, or more. The cross-bars may be substantially perpendicular or transverse relative to the longitudinal axis of the guard rails 404. The guard rails 404 may be straight or substantially straight, as shown in FIGS. 8-10, or they may define one or more curved portions. Examples may also include one or more planar features or sides connecting or spanning between the guard rails 404 to further prevent tissue from obstructing the view of the treatment site.

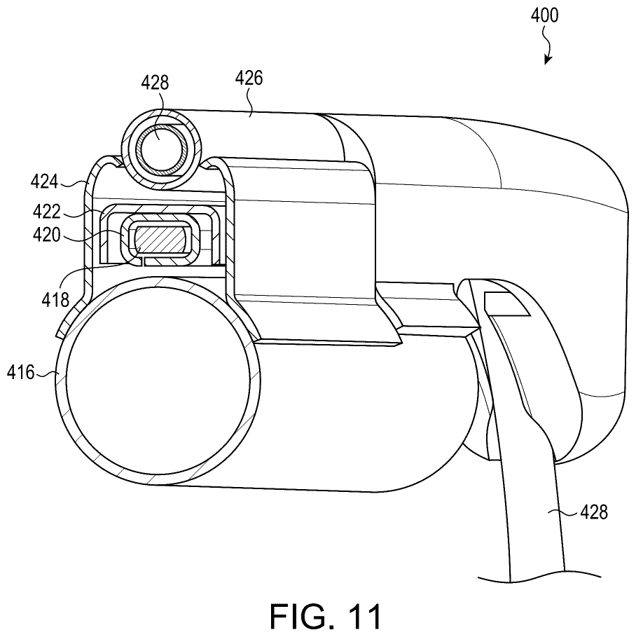

FIG. 11 provides a cross-sectional perspective view of a distal portion of the shaft assembly 400, showing the scope tube 416, UE pusher member 418, UE channel 420, cutter member 422, shaft housing 424, needle tube 426, and needle 428. The guard rails 404a, 404b are not shown for ease of illustration. The scope tube 416 is configured to receive and accommodate longitudinal movement of a scope, and the UE pusher member 418 is configured to push each UE distally through the UE channel 420 for seating onto a tensioned suture. A distal end of the cutter member 422 cuts the tensioned suture after UE attachment. The needle tube 426 receives and accommodates longitudinal movement of the needle 428 to and from a treatment site.

The depicted arrangement of shaft assembly components enhances and maintains visualization of the target site and the implant deployment processes, e.g., needle puncture and UE ejection and/or attachment. In particular, with the scope tube 416 positioned below both the UE channel 420 and needle tube 426 (or above, depending on the orientation of the device), instead of sandwiched between the UE channel 420 and needle tube 426, for example, visual obstruction of the distal portion of the shaft assembly is reduced and direct visualization of needle puncture and UE ejection and attachment is enabled.

A visualization system, such as that shown in FIGS. 8-11, may be coupled with a display device, e.g., user interface, configured to display the camera view in real time during the procedure. The user performing the procedure may adjust the positioning of the device and/or timing of implant deployment depending on the displayed view.

Examples of the visualization system represented in FIGS. 8-11 may be included in one or more of the devices shown and described herein, for example one or more devices having or compatible with a removable cartridge and/or one or more devices configured to simultaneously hold and serially deploy multiple implants. Accordingly, the visualization system may be featured in one or more devices configured to deploy multiple prostatic implants during a single procedure, which may occur without removing the delivery device or component thereof, e.g., the shaft assembly, from the patient.

Embodiments of the visualization system shown in FIGS. 8-11 may be included in a single-fire delivery device, which may be a device loaded with a single implant. The shaft assembly of such single-fire devices may require removal during a procedure to accommodate the exchange of a removable cartridge with the corresponding handle assembly or to accommodate the insertion into a patient of an entirely separate device (which may lack a removable cartridge) containing another implant. Non-limiting examples of such devices are described in U.S. Pat. No. 11,298,115 and US Patent Application Publication No. 2021/0378658, both of which are incorporated by reference herein. Accordingly, the visualization system depicted in FIGS. 8-11 may be incorporated into a variety of implant delivery devices, non-limiting examples of which may include, but are not limited to, the devices, device components, and device assemblies described herein. Incorporation of one or more embodiments of the disclosed visualization system may provide enhanced viewing relative to preexisting visualization systems.

Fluid irrigation at the treatment site may also be important for creating and maintaining an unobstructed view of one or more implant delivery steps disclosed herein during an implant delivery procedure. Fluid irrigation may also be important for effective performance of the procedure, as unwanted debris may impede one or more steps of implant deployment, including needle insertion into the targeted tissue and ejection of each UE. Accordingly, methods of delivering one or more implants to a prostate gland, for example using one or more of the devices and/or device components disclosed herein, may involve irrigating the treatment site during the procedure with a fluid irrigant such as water or saline. Irrigating the treatment site, which may include at least a portion of a prostatic urethra, may be important for maintaining a clear view of the treatment site by washing away tissue debris and blood, for instance.

Irrigation fluid may be pressurized, for instance via gravity or a pump, to expand the prostatic urethra for proper visualization during a procedure described herein. Fluid evacuation may also relieve pressure on a subject's bladder. Effective irrigation of the treatment site may therefore address a variety of issues that may arise during a procedure. Addressing one or more of these issues may be especially important during a procedure in which multiple implants are deployed. For example, maintaining adequate visualization of the treatment site during a multi-implant delivery procedure in which the shaft assembly is not removed from the patient between implant deployments may be difficult given that the fluid used in the patient during the procedure will likely become full of blood and tissue that may obscure the user's view captured using a viewing device, such as an endoscope or cystoscope, which may be provided as a scope assembly in or coupled with the elongate shaft assembly.

Embodiments disclosed herein may also include delivery devices, components, assemblies, and/or systems configured to irrigate a treatment site during an implant procedure. Examples of such embodiments may be controllable by a user, such that the introduction and removal of fluids to and from the urethra and/or bladder may be controlled in real time in response to user input. Accordingly, the irrigation systems described herein may comprise user-activated, controllable fluid management systems.

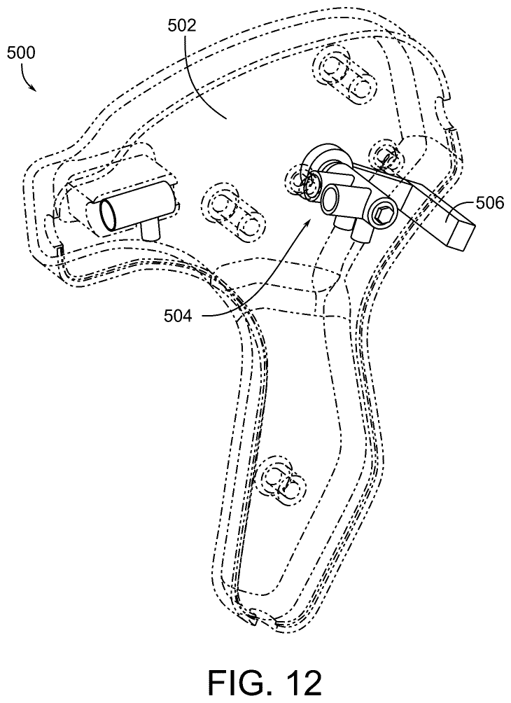

Embodiments of the delivery devices disclosed herein, such as delivery device 500 shown in FIG. 12 (or more specifically, the handle assembly thereof), may be configured to replace irrigation fluid in the visual field at the treatment site, within the prostatic urethra, in a manner controllable by a user. In some embodiments, a device may include an additional lumen that extends from a back portion of the handle assembly to the distal tip of the elongate shaft assembly, near a portion of the device featuring a camera for visualization, e.g., an endoscope or cystoscope. The lumen may be directly or indirectly attached to an external fluid source, which may be suspended on an IV pole or pressured by a pump. The lumen may be configured to serve as the fluid inflow to the patient. For fluid outflow, a lumen may be attached to a sheath that may be introduced into the urethra in some embodiments. According to such embodiments, the sheath may be reusable or it may be a component of the delivery device itself. In examples featuring a reusable sheath, the fluid may be re-routed into the handle assembly of the device for control.

Embodiments of a delivery device featuring an irrigation system may also include one or more user-accessible controls coupled or integrated with the device to manage fluid inflow and outflow. Examples of such controls may include one or more actuators, buttons, switches, levers, or stop cocks configured to regulate both the starting and stopping of fluid flow. Examples may also be configured to adjust a fluid flow rate. Embodiments may also include one or more valves, which may be included in the handle assembly, the valves coupled with one or more actuators and configured to open and close in response to user engagement with the actuators. The actuators, e.g., buttons or stop cocks, may be combined into a single actuator, actuator section, or actuator assembly to control two or more valves in some embodiments, which may allow the user to quickly turn the flow on and off during a procedure. Such a configuration may also enable the implementation of a continuous, constant flow rate that may be adjustable to the user's preferences. Both inflow and outflow may be controlled simultaneously.

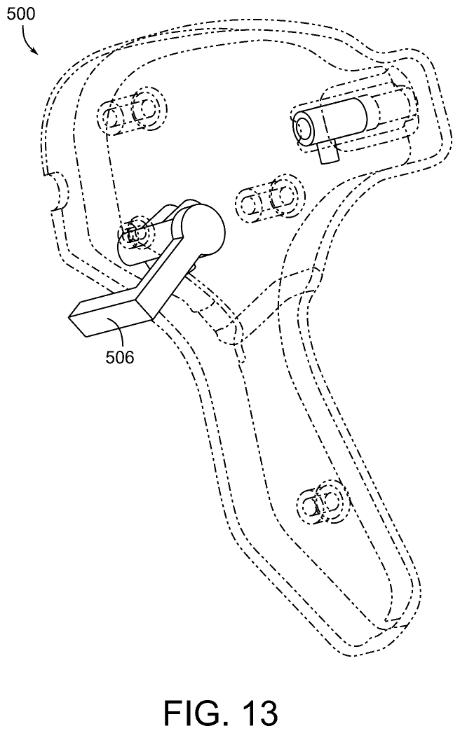

FIG. 12 provides a partially transparent view of a portion of a handle assembly 502 of the delivery device 500. The delivery device shown in this embodiment includes a stop cock component or assembly 504, which in this embodiment features two stop cocks. The manually engageable actuator, here lever 506, is coupled with the stop cock assembly 504 and configured to control the “on” and “off” of fluid flow to the treatment site in response to user engagement. FIG. 13 provides another partially transparent view of the handle assembly 502, showing the lever 506 protruding from a side/rear of the device, where it may be actuated by a user, e.g., via thumb press, during a treatment procedure. The irrigation flow rate may be set based on the sweep of the valve coupled with the manually engageable portion of the lever 506.

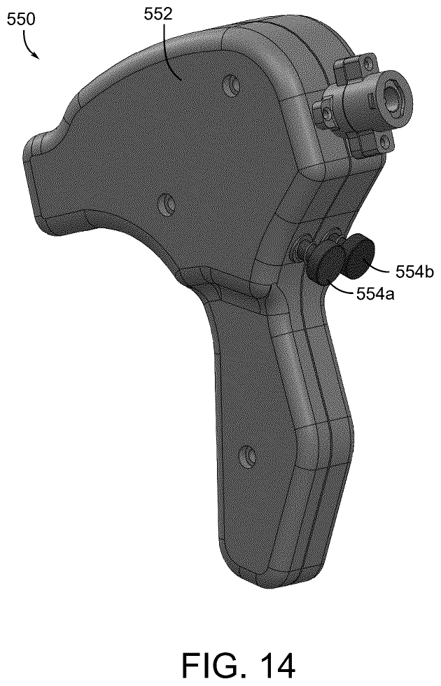

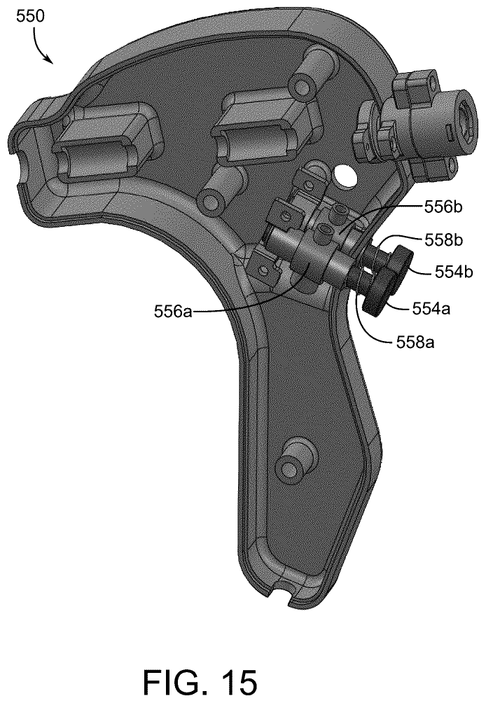

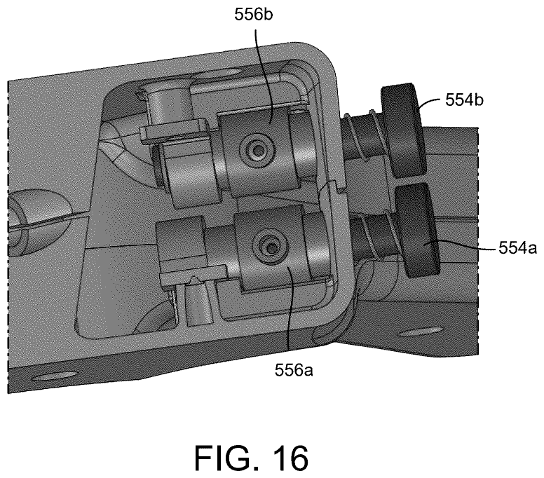

Another embodiment of a fluid control system may include spring-activated valves, e.g., trumpet valves, of which there may be two in some examples. Such configurations of a device may enable a user to depress, turn, or push an actuator of the device to selectively open a fluid inlet and/or outlet and adjust the fluid level or pressure in the patient during the procedure. Where two actuators are included, they may be pressed simultaneously to allow simultaneous activation that will allow flow through the device and anatomy, acting as a flush cycle. FIG. 14 illustrates an embodiment of such a delivery device 550 (or more specifically, the handle assembly without the elongate shaft assembly shown), which features a handle assembly 552 and two independent, user-engageable actuators in the form of buttons 554a, 554b configured to control the irrigation flow rate. One of the buttons may be configured to control the fluid inflow and the other may be configured to control the outflow. FIG. 15 provides a transparent view of the delivery device 550, showing the valves 556a, 556b and respective springs 558a, 558b connected respectively to buttons 554a, and 554b within the handle assembly. FIG. 16 provides a close-up, cutaway view of the valves 556a,b, which are trumpet valves in this particular, non-limiting example.

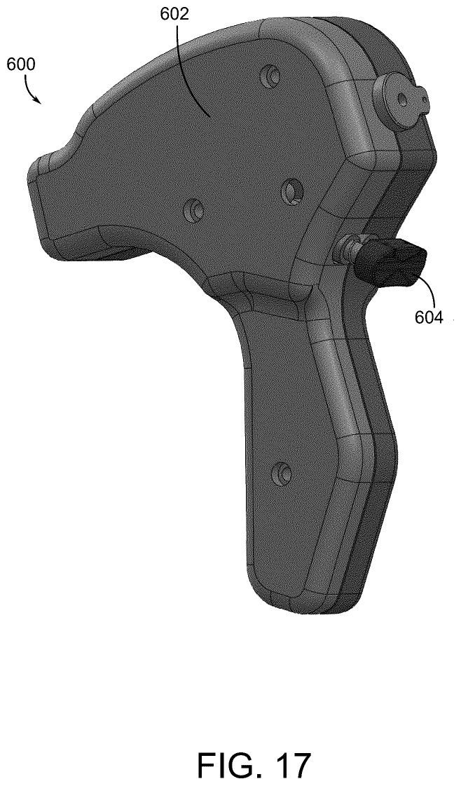

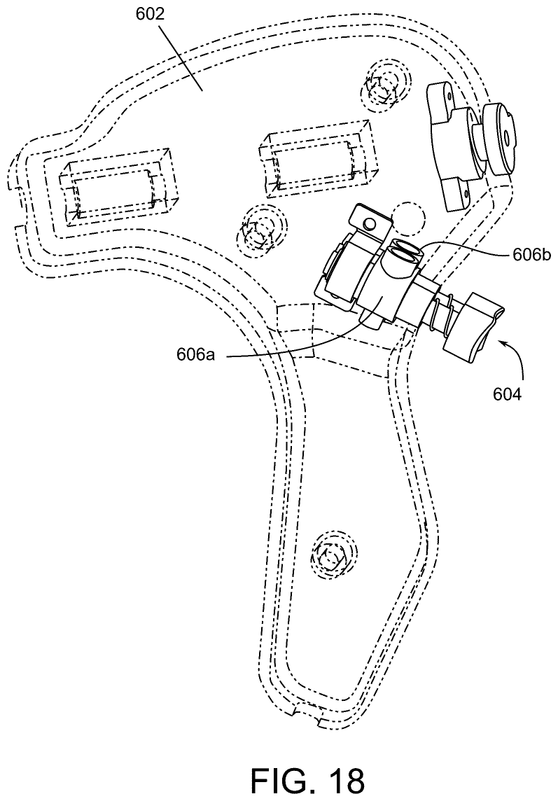

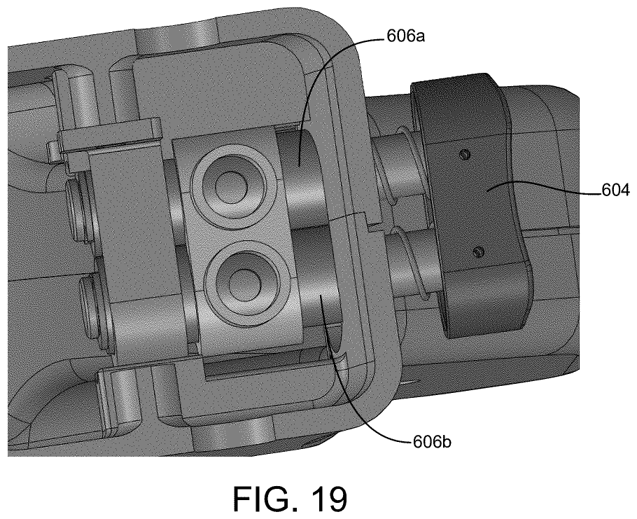

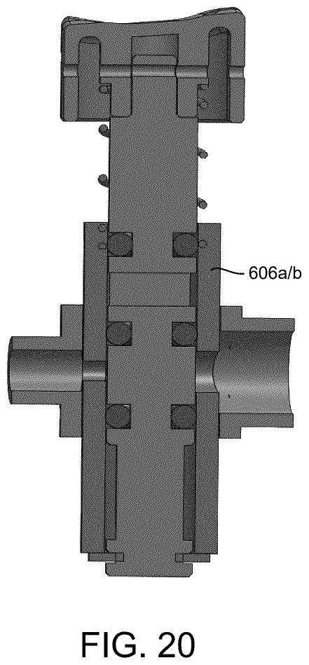

Embodiments may also include a single actuator, button or input-receiving element configured to control both the fluid inflow and outflow simultaneously. An example is shown in FIG. 17, which depicts a delivery device 600 comprising a handle assembly 602 and a manually engageable actuator comprising a button 604 extending from a rear portion of the handle assembly. In accordance with such embodiments, the button 604 may be pressed in a patterned manner, e.g., pulsed, to activate discrete flushing cycles. The button(s) (or other manually engageable components) may also be slightly pressed or feathered to allow for constant flow, with the rate dependent on how much the button (or activation assembly) is pressed or otherwise actuated. FIG. 18 provides a partially transparent view of the handle assembly 602, showing two valves 606a, 606b coupled with the externally protruding button 604, with one valve configured to control inflow and the other button configured to control outflow. A magnified top cutaway view of the single-actuator 604 irrigation control system is shown in FIG. 19. A cross-sectional view of a valve 606a or 606b is shown in FIG. 20.

Accordingly, one or more embodiments of the delivery devices disclosed herein may include a hand-held irrigation system configured to respond directly to user input during a procedure. Examples may be configured to adjust an irrigation rate in real time, at the direction of the user, to set and maintain a constant flow rate and/or to adjust the flow rate, again during the procedure. A variety of irrigation schemes may be employed, for example involving a constant, continuous flow rate that may be turned on and off, one or more flush cycles, or one or more periods of variable flow rate irrigation, one or more of which may be controlled by a user during a procedure. One or more actuators configured to receive user inputs may be included with a device, for example in the form of one or more buttons, dials, switches, toggles, knobs, levers, triggers, or otherwise movable or manipulatable components. The disclosed irrigation systems may enable a user to complete a procedure faster by maintaining visualization and supplanting the need to remove the device and flush the anatomy to maintain visualization. Use of a disclosed irrigation system may reduce the pressure and/or fluid volume required during a procedure, which may also improve patient comfort.

An irrigation system, such as one or more of those shown in FIGS. 12-20, may be coupled with a display device, e.g., a user interface, configured to display a scope view within the prostatic urethra during a procedure. The user performing the procedure may adjust the positioning of the device and/or timing of implant deployment depending on the displayed view.

Examples of the irrigation system represented in FIGS. 12-20 may be included in one or more delivery devices, for example one or more devices having or compatible with a removable cartridge and/or one or more devices configured to simultaneously hold and serially deploy multiple implants. Accordingly, the irrigation system may be featured in one or more devices configured to deploy multiple prostatic implants during a single procedure, which may occur without removing the delivery device or component thereof, e.g., the shaft assembly, from the patient.

Embodiments of the irrigation system shown in FIGS. 12-20 may also be included in a single-fire delivery device, which may be a device loaded with a single implant. The shaft assembly of such single-fire devices may require removal during a procedure to accommodate the exchange of a removable cartridge with the corresponding handle assembly or to accommodate the insertion into a patient of an entirely separate device (which may lack a removable cartridge) containing another implant. Non-limiting examples of such devices are described in U.S. Pat. No. 11,298,115 and U.S. Patent Application Publication No. 2021/0378658, the entire contents of which are incorporated by reference herein. Accordingly, the irrigation system depicted in FIGS. 12-20 may be incorporated into a variety of implant delivery devices, non-limiting examples of which may include, but are not limited to, the devices, device components, and device assemblies described herein. Incorporation of one or more embodiments of the disclosed irrigation system may provide enhanced viewing relative to preexisting irrigation systems.

In some examples, irrigation of a treatment site may be achieved using an elongate sheath device in addition to or in lieu of the irrigation systems shown in FIGS. 12-20. The sheath device may be provided together or separately from a delivery device, the latter including an elongate shaft assembly attached to a handle assembly. The inner lumen defined by the sheath device may receive and accommodate longitudinal movement, e.g., sliding, of the shaft assembly during a treatment procedure. Methods of compressing a tissue, such as prostate tissue, may thus involve delivering one or more implants to the tissue while irrigating the prostatic urethra with one or more of the disclosed irrigation systems. In some examples, an irrigation system may include an elongate sheath device, as described below, and associated methods may involve inserting the elongate sheath device into the urethra prior to inserting the elongate shaft assembly of an implant delivery device into the urethra.

FIG. 21 shows an example of an elongate sheath device configured to irrigate (or facilitate or contribute to the irrigation of) a treatment site within a subject. The sheath device 700 may be used to inject and withdraw irrigation fluid (together with a delivery device) from the treatment site, which may span all or at least a portion of the visual field of the treatment site, e.g., within the prostatic urethra. The sheath device 700 may be configured to receive a shaft assembly of a delivery device, for example within an elongate, inner lumen defined by the sheath device 700, spanning from its proximal end to its distal end. Together, the sheath device 700 and delivery device can circulate fluid into and out of a patient, the outflow further containing blood and debris.

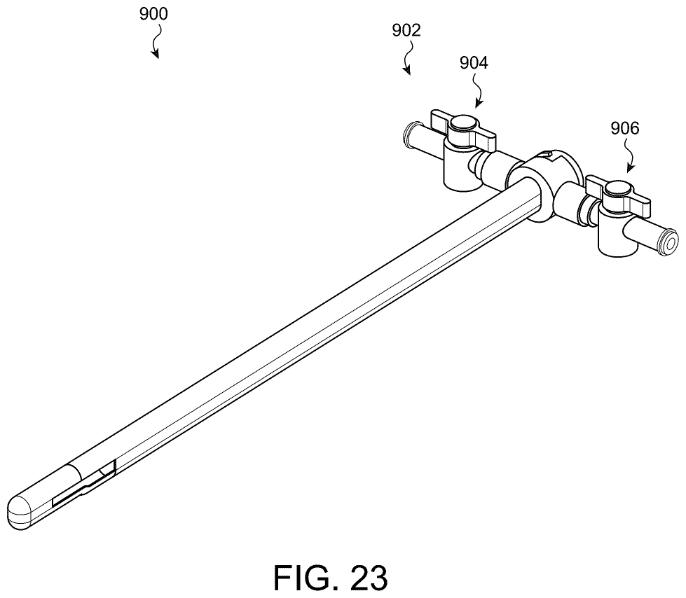

The proximal end 702 of the sheath device 700 device may be attached to or include a sheath hub 704, which can include one or more ports 706, 708, one or both of which can be an inflow port configured to receive an irrigation fluid (e.g., water or saline) by mating with a fluid injection line or device, e.g., tube, which may be connected to a fluid source, e.g., IV bag. The ports can be female luer-like ports located on the sides of the device to allow for connections with male luer connectors, allowing fluid routing. Stopcocks may additionally or alternatively be used for fluid routing (see e.g., FIG. 23). The stopcocks may be directly integrated for irrigation controls. The sheath device 700, including the sheath hub 704, may be plastic and disposable in some examples.

Extending from the sheath hub 704 is an elongate shaft of the sheath device 700, referred to herein as shaft portion 710. The distal end 712 of the sheath device 700 includes one or more cutouts, window portions, or openings 714. Embodiments of the shaft portion 710 may have a cuboid cross-sectional shape and an atraumatic, e.g., rounded, distal tip. One or more, e.g., two, cutouts or openings 714 may be located along the sides of the shaft portion 710, with the distal-most opening being at the bottom of the shaft portion 710 (e.g., relative to the upright orientation of the associated handle assembly), allowing for the needle of the associated delivery device to pass or sweep through the shaft portion unimpeded, and for an implant to eject. These features enable a plastic sheath to fully encapsulate the metal shaft components of the delivery device's shaft assembly. The sheath device 700 may also provide atraumatic benefits over the existing reusable sheaths, e.g., 20-Fr sheaths, by enabling a smooth, transition-less interface with surrounding tissue.

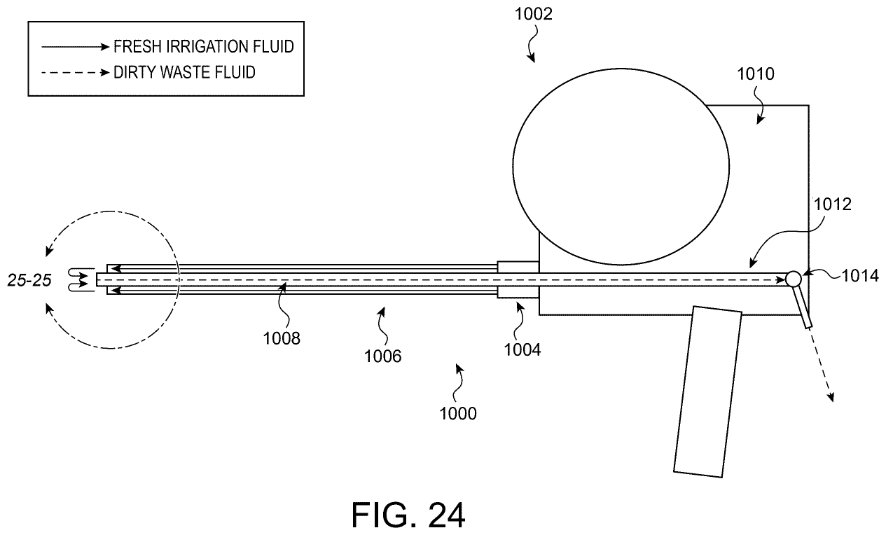

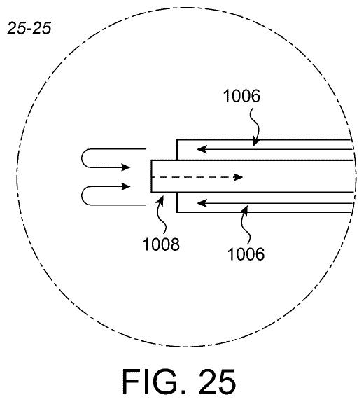

The proximal end 702 of the sheath device 700 may include a connector portion 716, which may comprise a male thread-like feature configured to connect to an associated delivery device and/or visual obturator, both of which can have a locking mechanism that engages with the thread-like feature of the connector portion 716 to lock the delivery device to the sheath device 700. Upon engagement of the sheath device 700 with a delivery device, continuous passive outflow of the irrigation fluid and any debris contained therein is enabled. As further detailed in FIGS. 24 and 25, fresh irrigation fluid flows through the sheath to the target site. Debris and blood are then flushed out through irrigation channels in the delivery system shaft assembly. An inflow channel in the sheath device 700 may be isolated from the outflow channel in the shaft assembly of the separate delivery device. This results in directional fluid flow that enables fluid circulation at the distal end of the delivery device, in front of the cystoscopy lens, promoting improved field-of-view clearing relative to preexisting devices.

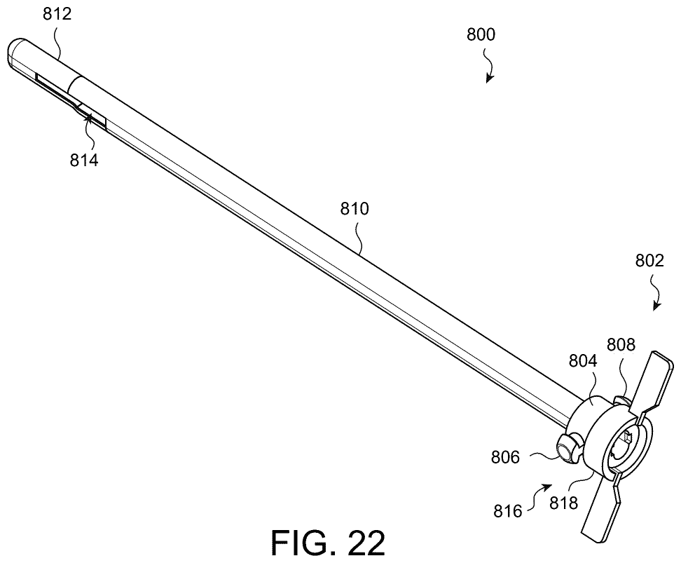

FIG. 22 shows another example of an elongate sheath device 800 configured to irrigate (or facilitate or contribute to the irrigation of) a treatment site within a subject. Like sheath device 700, sheath device 800 may be used to inject and withdraw (with a delivery device) irrigation fluid from a treatment site, which may span all or at least a portion of the visual field of the treatment site, e.g., within the prostatic urethra. The sheath device 800 also includes a proximal end 802 attached to or including a sheath hub 804, which can include one or more ports 806, 808, one or both of which can be an inflow port configured to receive an irrigation fluid, e.g., water or saline, by mating with a fluid injection line or device, e.g., tube, which may be connected to a fluid source, e.g., IV bag. The ports can be female luer-like ports located on the sides of the device to allow for connections with male luer connectors, allowing fluid routing. The sheath hub 804 may be plastic in some examples.

Extending from the sheath hub 804 is an elongate shaft portion 810, which may define an inner lumen that extends from the proximal end to the distal end of the sheath device 800. The distal end 812 of the sheath device 800 includes one or more cutouts, window portions, or openings 814. Embodiments of the shaft portion 810 may have a cuboid cross-sectional shape and an atraumatic, e.g., rounded, distal tip. One or more, e.g., two, of the cutouts or openings may be located along the sides of the shaft portion, with the distal-most opening being at the bottom of the shaft portion (relative to the upright orientation of the associated handle assembly), allowing for the needle of the associated delivery device to pass or sweep through the sheath portion unimpeded pursuant to implant ejection and delivery.

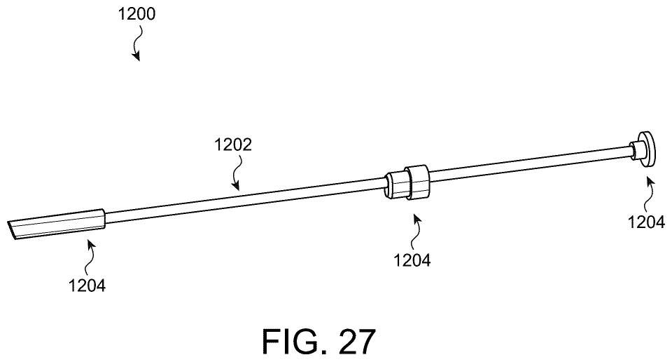

Accordingly, sheath device 800 may be similar or identical to sheath device 700 in many respects. A difference between the two devices is the connector portion or mechanism for coupling or attaching the sheath devices to an associated delivery device. In particular, relative to sheath device 700, sheath device 800 is configured to invert the interaction with the locking mechanism on the sheath and the male thread-like feature on the delivery device and visual obturator (see FIG. 27). The connection mechanism 816 of sheath device 800 may include or couple with an adaptor member 818 configured to couple the sheath device 800 to an associated delivery device. In some examples, the adaptor 818 is formed with, or constitutes a component of, a delivery device, e.g., the handle assembly.

Upon engagement of the sheath device 800 with a delivery device, continuous passive outflow of the irrigation fluid and any debris contained therein is enabled. Fresh irrigation fluid flows through the sheath to the target site. Debris and blood are then flushed out through irrigation channels in the delivery system shaft assembly. An inflow channel in the sheath device 800 may be isolated from the outflow channel in the shaft assembly of the coupled delivery device. This results in directional fluid flow that enables fluid circulation at the distal end of the delivery device, in front of the cystoscopy lens, promoting improved field-of-view clearing relative to preexisting devices.

Unlike preexisting devices, the sheath devices disclosed herein may not be reusable in some embodiments. The sheath devices disclosed herein may not be made of stainless steel in some examples.

FIG. 23 provides a perspective view of another sheath device 900. As shown, the proximal end 902 of the device may include two stopcocks 904, 906 to facilitate and control fluid inflow.

FIG. 24 shows a schematic of an example of a sheath device 1000 coupled to a delivery device 1002 in accordance with embodiments disclosed herein. The sheath device 1000 includes a sheath hub 1004 (configured to receive fluid inflow, e.g., from an IV bag) and an elongate shaft portion 1006. Within the shaft portion 1006 extends an elongate shaft assembly 1008 of the delivery device 1002. The handle assembly 1010 of the delivery device 1002 includes an outflow channel 1012 and outflow port 1014. Together, the sheath device 1000 and delivery device 1002 are configured to drive directional fluid flow. In particular, fresh irrigant fluid flows through an elongate lumen of the sheath device 1000 (see solid arrows) to the distal tip of the assembly. Debris and blood are then removed from the treatment site by flowing through the elongate lumen of the shaft assembly 1008 of the delivery device 1002 (see dashed arrow), eventually passing through the channel 1012 of the handle assembly 1010 and flushed out through the outflow port 1014. Directional flow marks an improvement over preexisting devices, promoting fluid circulation in front of the camera lens at the distal tip of the delivery device, thereby also improving visualization during a treatment procedure.

FIG. 25 provides a close-up view of a distal end of the shaft portion 1006 of the sheath device 1000 and the shaft assembly 1008 of the delivery device 1002, showing Detail 25-25 of FIG. 24, showing the directional flow of fresh irrigant distally through and out of the distal end of the shaft portion 1006 (see solid arrows), and the redirected outflow of blood/debris/irrigant proximally back through the lumen defined by the shaft assembly 1008 (see dashed arrow). The cross-sectional diameter of the shaft portion 1006 of the sheath device 1000 is greater than that of the shaft assembly 1008, and the fresh irrigant may flow distally through the devices within the space between the outer surface of the shaft assembly 1008 and inner surface of the shaft portion 1006 of the sheath device 1000.

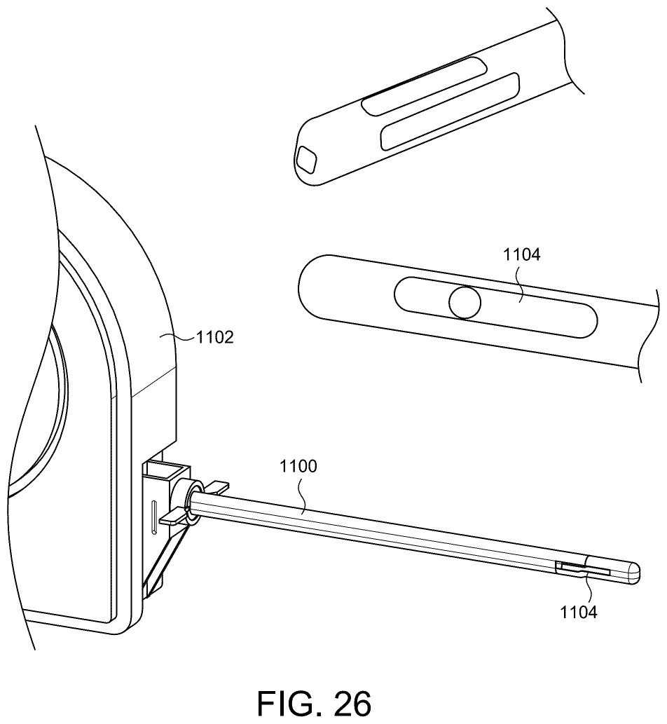

FIG. 26 provides views of a shaft portion 1100 of a sheath device coupled to a delivery device 1102. As shown, the shaft portion 1100 may have a smooth, transitionless, and atraumatic design configured to reduce tissue trauma and injury when being advanced through a patient's urethra. The sheath portion 1100 may fully enclose the metal components of a shaft assembly positioned therein. The distal windows 1104 of the sheath device 1100 may be defined or surrounded by transparent material, e.g., plastic, to maximize visibility at the treatment site and thus the success of implant deployment.

FIG. 27 provides views of a visual obturator 1200 compatible with the devices disclosed herein. As shown, the visual obturator can include or comprise a hypotube 1202 and plastic members or components 1204, which may be manually engageable, along the length of the tube. The visual obturator 1200 may be a single-use device configured to attach and detach seamlessly with a sheath device and scope. The visual obturator 1200 may be made of plastic in some embodiments. The visual obturator 1200 may be a passive component that may replace existing, reusable visual obturators. The visual obturator 1200 may be necessary to include for compatibility with multi-fire implant delivery devices or components thereof, e.g., shaft assemblies, sheath devices, etc.

Examples of the irrigation systems represented in FIGS. 21-27 may be included in one or more delivery devices, for example one or more devices having or compatible with a removable cartridge and/or one or more devices configured to simultaneously hold and serially deploy multiple implants. Accordingly, the irrigation systems may be featured in one or more devices configured to deploy multiple prostatic implants during a single procedure (e.g., multi-fire delivery devices), which may occur without removing the delivery device or component thereof, e.g., the shaft assembly, from the patient.

Embodiments of the irrigation systems shown in FIGS. 21-27 may also be included in a single-fire delivery device, which may be a device loaded with a single implant. The shaft assembly of such single-fire devices may require removal during a procedure to accommodate the exchange of a removable cartridge with the corresponding handle assembly or to accommodate the insertion into a patient of an entirely separate device (which may lack a removable cartridge) containing another implant. Non-limiting examples of such devices are described in U.S. Pat. No. 11,298,115 and U.S. Patent Application Publication No. 2021/0378658, the entire contents of which are incorporated by reference herein. Accordingly, the irrigation systems depicted in FIGS. 21-27 may be incorporated into a variety of implant delivery devices, non-limiting examples of which may include, but are not limited to, the devices, device components, and device assemblies described herein. Incorporation of one or more embodiments of the disclosed irrigation system may provide enhanced viewing relative to preexisting irrigation systems.

### EXAMPLES

The above Detailed Description includes references to the accompanying drawings, which form a part of the Detailed Description. The Detailed Description should be read with reference to the drawings. The drawings show, by way of illustration, specific embodiments in which the present accessory devices and associated methods can be practiced. These embodiments are also referred to herein as “examples.”

From the foregoing it will be appreciated that, although specific embodiments of the invention have been described herein for purposes of illustration, various modifications may be made without deviating from the spirit and scope of the invention.

The disclosed devices, systems, assemblies, and components thereof can be implemented in various treatment devices employed for various medical purposes including, but not limited to, retracting lifting, compressing, approximating, supporting, remodeling, repositioning, ablating, or otherwise altering tissues, organs, anatomical structures, grafts, or other material found within the body of a human or animal subject. In certain embodiments, treatment devices are intended to displace, compress, retract, or destroy tissue of the prostate to facilitate treatment of disease or disorders, such as BPH.

The Detailed Description is intended to be illustrative and not restrictive. For example, the above-described examples (or one or more features or components thereof) can be used in combination with each other. For example, one or more visualization components, systems, or assemblies disclosed herein may be combined with one or more irrigation components, systems, or assemblies disclosed herein. One or more of the irrigation and/or visualization components, systems, and/or assemblies may also be combined with one or more implant delivery devices or components thereof disclosed herein. Other embodiments can be used, such as by one of ordinary skill in the art upon reviewing the above Detailed Description. Also, various features or components have been or can be grouped together to streamline the disclosure. This should not be interpreted as intending that an unclaimed disclosed feature is essential to any claim. Rather, inventive subject matter can lie in less than all features of a particular disclosed embodiment. Thus, the following claim examples are hereby incorporated into the Detailed Description, with each example standing on its own as a separate embodiment:

In Example 1, a delivery device configured to deploy one or more implants to a prostate gland of a patient includes a handle assembly and an elongate shaft assembly. The delivery device may also include an irrigation component, system, or assembly configured to control an irrigation fluid inflow and/or outflow to and from a treatment site within a prostatic urethra during a treatment procedure in response to user input.

In Example 2, at least a portion of the irrigation component, system, or assembly of Example 1 may be included in or coupled with the elongate shaft assembly.

In Example 3, the delivery device of one or both of Examples 1 or 2 may further include a scope assembly configured to view a distal end of the shaft assembly during a medical procedure involving the deployment of the one or more implants to the prostate gland of a patient.

In Example 4, the handle assembly of any one or any combination of Examples 1-3 may include at least one manually engageable actuator configured to control the irrigation fluid inflow and/or outflow to and from a treatment site within a prostatic urethra during a treatment procedure in response to user input.

In Example 5, the at least one manually engageable actuator of Example 4 may include one or more buttons or levers.

In Example 6, the at least one manually engageable actuator of Example 4 or 5 may include two actuators, a first actuator configured to control a fluid inflow rate, and a second actuator configured to control a fluid outflow rate.

In Example 7, the at least one actuator of any one or any combination of Examples 4-6 may be attached to or coupled with one or more valves or a valve assembly within the handle assembly.

In Example 8, the one or more valves of Example 7 may be spring-activated or loaded.

In Example 9, the at least one actuator of any one of Examples 4-7 may be coupled with one or more stop cocks or a stop cock assembly within the handle assembly.

In Example 10, a constant flow rate may be implemented by engaging the at least one actuator of any one or any combination of Examples 1-9, wherein the constant flow rate may be adjusted by adjusting a level of engagement with the actuator, wherein adjusting the level of engagement is determined by an amount in which the actuator is pushed or swept by a user.

In Example 11, the delivery device of any one or any combination of Examples 1-10 may further include a removable cartridge, the removable cartridge configured to contain at least one distal anchor component and a suture attached thereto (“CT/suture”) of an implant, the distal anchor component configured to anchor to a prostatic capsule of the prostate gland, and the suture configured to be placed within the prostate gland.

In Example 12, the handle assembly of any one or any combination of Examples 1-10 may be configured to contain at least one distal anchor component and a suture attached thereto (“CT/suture”) of an implant, the distal anchor component configured to anchor to a prostatic capsule of the prostate gland, and the suture configured to be placed within the prostate gland.

In Example 13, the handle assembly of any one or any combination of Examples 1-12 may further include or be coupled with a needle configured to pierce the prostate gland and accommodate at least a portion of an implant therein.

In Example 14, at least a portion of the elongate shaft assembly of Example 13 may be configured to be advanced longitudinally through the urethra of the patient while accommodating advancement of the needle therethrough.

In Example 15, the handle assembly of any one or any combination of Examples 1-14 may further include at least one actuator configured to drive, initiate, or cause deployment of the one or more implants in response to user engagement.

In Example 16, a delivery device configured to deploy one or more implants to a prostate gland of a patient may include a handle assembly and an elongate shaft assembly. The delivery device may further include a visualization component, system, or assembly configured to provide a real-time view of, generate an image of, and/or obtain a live recording or video feed of a treatment site.

In Example 17, at least a portion of the visualization component, system, or assembly of Example 16 may be included in or coupled with the elongate shaft assembly.

In Example 18, the visualization component, system, or assembly of one or both of Examples 16 and 17 may include a scope assembly configured to view a distal end of the shaft assembly during a medical procedure involving the deployment of the one or more implants to a prostate gland of a patient.

In Example 19, the scope assembly of Example 18 may include an endoscope or cystoscope, wherein at least a distal portion thereof is positioned adjacent an exit port at a distal portion of the shaft assembly.

In Example 20, the delivery device of any one or any combination of Examples 16-19 may further include an elongate needle configured to extend through the exit port, wherein the distal end of the scope assembly is configured to view the exit port.

In Example 21, the scope assembly of any one or any combination of Examples 16-20 may be configured to view an attachment of a urethral endpiece to a suture, as shown and described herein.

In Example 22, the delivery device of any one or any combination of Examples 16-18 may further include one or more structures, segments, portions, or guard rails configured to span across the exit port.

In Example 23, the one or more structures, segments, portions, or guard rails of Example 22 may be configured to prevent prostatic tissue from obstructing a view of the exit port.

In Example 24, the one or more structures, segments, portions, or guard rails of one or both of Examples 22 and 23 may include two guard rails defining a space therebetween.

In Example 25, the space of Example 24 may be configured to accommodate passage of a penetrating member or needle therethrough.

In Example 26, a method for viewing and/or irrigating a treatment site within a prostatic urethra during a prostatic implant delivery process may involve using any one or any combination of the delivery devices, systems, and/or assemblies of Examples 1-25.

In Example 27, a method for deploying one or more implants to one or more lobes of a prostate gland may involve one or more of the following steps: 1) positioning an elongate portion of a delivery device disclosed herein in a prostatic urethra; 2) advancing a penetrating member or needle coupled with at least a portion of a prostatic implant through the elongate portion of the delivery device, the prostatic implant comprising a distal anchor portion connected to a middle portion; 3) penetrating a lobe of the prostate gland with the distal tip of the needle; 4) advancing the needle through the lobe of the prostate gland until the distal tip is positioned outside a prostatic capsule of the lobe; 5) unsheathing the prostatic implant by retracting the needle in a proximal direction; 6) tensioning the middle portion; and 7) securing a proximal anchor to the middle portion.

In Example 28, the method of Example 27 may further involve repositioning the elongate portion of the delivery device in the prostatic urethra and repeating steps 2-7 to deploy a second implant in the prostate gland.

In Example 29, one or more of steps 2-7 of Example 27 may be performed by a user engaging one or more actuators on the delivery device.

In Example 30, the method of any one or any combination of Examples 27-29 may further involve viewing, generating an image of, and/or obtaining a live recording or video feed of the prostatic urethra.

In Example 31, viewing, generating an image of, and/or obtaining a live recording or video feed of the prostatic urethra may involve utilizing a visualization system, component, or assembly recited in any one or any combination of Examples 16-25.

In Example 32, the method of any one or any combination of Examples 27-31 may further involve irrigating a treatment site with an irrigation fluid.

In Example 33, the irrigating of Example 32 may involve engaging one or more actuators on the delivery device, the one or more actuators configured to control the inflow and/or outflow of the irrigation fluid to a treatment site within the prostatic urethra in response to user engagement.

In Example 34, the irrigating of one or both of Examples 32 and 33 may involve establishing or maintaining a continuous inflow and/or outflow of the irrigation fluid to and/or from the treatment site.

In Example 35, the irrigating of any one or any combination of Examples 32-35 may involve initiating one or more flush cycles.

In Example 36, the delivery device utilized in accordance with any one or any combination of Examples 27-35 may further include a fluid inlet and/or outlet configured to accommodate passage of an irrigation fluid therethrough.

In Example 37, a system configured to deploy one or more implants to a prostate gland of a patient includes a handle assembly and an elongate shaft assembly fixed or attached thereto. The system also includes an elongate sheath device defining an inner lumen configured to receive the elongate shaft assembly, the elongate sheath device configured to direct an irrigant to a treatment site.

In Example 38, the elongate shaft assembly of Example 37 may be configured to accommodate outflow of blood and debris from the treatment site.

In Example 39, the elongate sheath device of one or both of Examples 37 and 38 may be attached to or includes a proximal hub, an elongate sheath portion, and one or more window features at a distal end of the sheath device.

In Example 40, the proximal hub of Example 39 may include two ports configured to receive the irrigant from a fluid source.

In Example 41, the proximal hub of Example 39 may include or is attached to two or more stopcocks configured to control irrigant inflow from a fluid source into the sheath portion.

In Example 42, the sheath device of any one or any combination of Examples 37-41 may include a connector portion configured to couple a proximal end of the sheath device to the handle assembly.

In Example 43, the connector portion of Example 42 comprises a threaded surface.

In Example 44, the connector portion of one or both of Examples 42 and 43 may include a male thread-like feature.

In Example 45, the handle assembly of any one or any combination of Examples 42-44 may include a locking mechanism configured to engage the connector portion to lock the delivery device to the sheath device.

In Example 46, a cross-sectional gap between an outer surface of the elongate shaft assembly and an inner surface of the elongate sheath portion of any one or any combination of Examples 37-46 may define a fluid inflow channel.

In Example 47, the delivery device of any one or any combination of Examples 37-46 may be configured to enable continuous passive outflow of contaminated irrigant, blood, and/or debris.

In Example 48, the elongate sheath portion of any one or any combination of Examples 37-47 may define a cuboid or approximately cuboid cross-sectional shape.

In Example 49, a distal tip of the elongate sheath portion of any one or any combination of Examples 37-48 may be rounded or curved.

In Example 50, the handle assembly of any one or any combination of Examples 37-49 may include a fluid outflow channel in fluid communication with the elongate shaft assembly.

In Example 51, the handle assembly of any one or any combination of Examples 37-50 may include a fluid outflow port.

In Example 52, the system of any one or any combination of Examples 37-51 may further include a visual obturator.

In Example 53, the system of any one or any combination of Examples 37-52 may further include a scope assembly configured to view a distal end of the shaft assembly.

In Example 54, the system of any one or any combination of Examples 37-53 may further include a removable cartridge, the removable cartridge configured to contain at least one distal anchor component and a suture attached thereto (“CT/suture”) of an implant, the distal anchor component configured to anchor to a prostatic capsule of the prostate gland, and the suture configured to be placed within the prostate gland.

In Example 55, the system of any one or any combination of Examples 37-53 may include a handle assembly configured to contain at least one distal anchor component and a suture attached thereto (“CT/suture”) of an implant, the distal anchor component configured to anchor to a prostatic capsule of the prostate gland, and the suture configured to be placed within the prostate gland.

In Example 56, the handle assembly of one or both of Examples 54 and 55 may further include or be coupled with a needle configured to pierce the prostate gland and accommodate at least a portion of an implant therein.

In Example 57, at least a portion of the elongate shaft assembly of any one or any combination of Examples 54-56 may be configured to be advanced longitudinally through the urethra of the patient while accommodating advancement of the needle therethrough.

In Example 58, the handle assembly of any one or any combination of Examples 54-57 may further include at least one actuator configured to drive, initiate, or cause deployment of one or more implants in response to user engagement.

In Example 59, a method for irrigating a treatment site within a prostatic urethra during a prostatic implant delivery process involves using any one or any combination of the systems, devices, and components of any one or any combination of Examples 37-58.

In Example 60, a sheath device configured to deliver an irrigant to a treatment site includes a proximal hub and an elongate shaft defining a lumen that extends an entire length of the shaft.

In Example 61, the sheath device of Example 60 may further include one or more inflow ports configured to receive an irrigant from a fluid source.

In Example 62, the sheath device of Example 60 may further include one or more stopcocks configured to control entry of an irrigant into the elongate shaft from a fluid source.

In Example 63, the sheath device of any one or any combination of Examples 60-62 may further include a connector portion configured to connect with a handle assembly of an implant delivery device.

In Example 64, a method for treating enlarged or inflamed tissue is implemented using any of the systems, devices, or components of any one or any combination of Examples 37-63.

In Example 65, a method for treating benign prostatic hyperplasia is implemented using any of the systems, devices, or components of any one or any combination of Examples 37-63.

In Example 66, a method for deploying one or more implants to one or more lobes of a prostate gland involves: 1) positioning an elongate portion of a delivery device disclosed herein in a prostatic urethra; 2) advancing a penetrating member or needle coupled with at least a portion of a prostatic implant through the elongate portion of the delivery device, the prostatic implant comprising a distal anchor portion connected to a middle portion; 3) penetrating a lobe of the prostate gland with the distal tip of the needle; 4) advancing the needle through the lobe of the prostate gland until the distal tip is positioned outside a prostatic capsule of the lobe; 5) unsheathing the prostatic implant by retracting the needle in a proximal direction; 6) tensioning the middle portion; and 7) securing a proximal anchor to the middle portion.

In Example 67, the method of Example 66 further involves irrigating the prostatic urethra with an irrigation fluid.

In Example 68, the method of one or both of Examples 66 and 67 may be implemented with any one or any combination of the systems, devices, and components of Examples 37-63.

In Example 69, the delivery device used in the method of Example 66 may be operatively coupled to an external fluid source.

In Example 70, the irrigation fluid used in any one or any combination of Examples 66-69 may include water and/or saline.

Example 71 involves compressing a prostate gland using any one or any combination of the systems, devices, and components of Examples 37-63.

In Example 72, the prostate gland of Example 71 may be enlarged relative to a healthy prostate gland.

Example 73 includes means for treating benign prostatic hyperplasia using any one or any combination of the systems, devices, and components of Examples 37-63.

Example 74 includes means for forming any one or any combination of the systems, delivery devices, assemblies, and/or components of any one or any combination of Examples 1-63.

In various examples and embodiments described herein, separate components may be considered or referred to as part of the same delivery device. For example, examples may feature a delivery device that includes a handle assembly and a removable cartridge. Such examples may also be considered or referred to as systems, such as, for example, a system that includes a handle assembly and a removable cartridge configured to couple therewith. The implant assemblies and components described herein, e.g., CT/suture assemblies and UEs, may similarly be considered components of a single delivery device or components of a system that includes a delivery device.

### CLOSING NOTES

Certain terms are used throughout this patent document to refer to particular features or components. As one skilled in the art appreciates, different people may refer to the same feature or component by different names. This patent document does not intend to distinguish between components or features that differ in name but not in function.

For the following defined terms, certain definitions shall be applied unless a different definition is given elsewhere in this patent document. The terms “a,” “an,” and “the” are used to include one or more than one, independent of any other instances or usages of “at least one” or “one or more.” The term “or” is used to refer to a nonexclusive or, such that “A or B” includes “A but not B,” “B but not A,” and “A and B.” All numeric values are assumed to be modified by the term “about,” whether or not explicitly indicated. The term “about” generally refers to a range of numbers that one of skill in the art would consider functionally equivalent to the recited value (e.g., having the same function or result). In many instances, the term “about” can include numbers that are rounded to the nearest significant figure. The recitation of numerical ranges by endpoints includes all numbers and sub-ranges within and bounding that range (e.g., 1 to 4 includes 1, 1.5, 1.75, 2, 2.3, 2.6, 2.9, etc. and 1 to 1.5, 1 to 2, 1 to 3, 2 to 3.5, 2 to 4, 3 to 4, etc.). The terms “patient” and “subject” are intended to include mammals, such as for human or veterinary applications. The terms “distal” and “proximal” are used to refer to a position or direction relative to the treating clinician. “Distal” and “distally” refer to a position that is distant from, or in a direction away from, the treating clinician. “Proximal” and “proximally” refer to a position that is near, or in a direction toward, the treating clinician.

The scope of the invention should be determined with reference to the appended claims, along with the full scope of equivalents to which such claims are entitled. In the appended claims, the terms “including” and “in which” are used as the plain-English equivalents of the respective terms “comprising” and “wherein.” Also, in the following claims, the terms “including” and “comprising” are open-ended; that is, a device, kit or method that includes features or components in addition to those listed after such a term in a claim are still deemed to fall within the scope of that claim. Moreover, in the following claims, the terms “first,” “second” and “third,” etc. are used merely as labels, and are not intended to impose numerical requirements on their objects.

The Abstract is provided to allow the reader to quickly ascertain the nature of the technical disclosure. It is submitted with the understanding that it will not be used to interpret or limit the scope or meaning of the claims.

What is claimed is:

1. A system configured to deploy one or more implants to a prostate gland of a patient, comprising:
a delivery device comprising:
a handle assembly attached to an elongate shaft assembly;
a visualization component configured to provide a real-time view of a treatment site inside a patient; and
one or more structures configured to span across an exit port defined by a distal portion of the elongate shaft assembly; and
an elongate sheath device defining an inner lumen configured to receive the elongate shaft assembly, the elongate sheath device configured to direct an irrigant to the treatment site.

2. The system of claim 1, wherein at least a portion of the visualization component is coupled with the elongate shaft assembly.

3. The system of 1, wherein the visualization component comprises a scope assembly configured to view the distal portion of the elongate shaft assembly at the treatment site.

4. The system of claim 3, wherein the scope assembly comprises an endoscope housed within a scope tube.

5. The system of claim 4, wherein a distal end of the endoscope is positioned proximal to the exit port.

6. The system of claim 1, further comprising an elongate needle configured to extend through the exit port.

7. The system of claim 1, wherein the one or more structures comprise rail members.

8. The system of claim 7, wherein the rail members are configured to prevent prostatic tissue from obstructing a view of the exit port.

9. The system of claim 8, wherein the rail members comprise two rail members defining a space therebetween.

10. The system of claim 1, wherein the treatment site comprises a prostatic urethra.

11. The system of claim 1, wherein the elongate shaft assembly is configured to accommodate outflow of blood and debris from the treatment site.

12. The system of claim 1, wherein the elongate sheath device includes a proximal hub.

13. The system of claim 12, wherein the proximal hub includes two ports configured to receive the irrigant from a fluid source.

14. The system of claim 12, wherein the proximal hub includes two or more stopcocks configured to control irrigant inflow from a fluid source.

15. The system of claim 1, wherein the elongate sheath device includes a connector portion configured to couple a proximal end of the elongate sheath device to the handle assembly.

16. The system of claim 15, wherein the connector portion comprises a threaded surface.

17. The system of claim 15, wherein the handle assembly includes a locking mechanism configured to engage the connector portion to lock the delivery device to the elongate sheath device.

18. The system of claim 1, wherein the elongate sheath device includes an elongate shaft portion, and wherein a cross-sectional gap between an outer surface of the elongate shaft assembly and an inner surface of the elongate shaft portion defines a fluid inflow channel.

19. The system of claim 1, wherein the delivery device is configured to enable continuous passive outflow of contaminated irrigant, blood, and/or debris.

20. The system of claim 1, wherein the handle assembly includes a fluid outflow channel in fluid communication with the elongate shaft assembly.
